SEQUENCE LISTING (19 sequences)

SEQ ID NO: 1 - DNA, 1083 bp, synthetic construct
  source 1..1083: mol_type = other DNA
atggctggccatctcgcaagtgacttcgcattttccccgcccccaggcggcggtggagatggacctggcggcccagaaccaggatgggtggacccccgcacgtggctttcttttcagggtccaccaggcggacccggtattggacccggcgtgggtcctgggtcagaagtctggggtatcccaccctgtcccccaccatacgaattttgcggcggaatggcctattgcggccctcaagtcggggtcggtctggtacctcagggtggtctggaaacatcccaaccagaaggtgaggccggtgtgggagttgaatccaattctgacggagcatctccagaaccttgtactgtgacaccaggagctgttaaattggagaaagaaaagctcgaacagaatccagaagaatcacaagatattaaggcgctccaaaaggagctggaacagttcgcgaaacttcttaaacagaaacgcattactctcgggtacacccaagcggacgttggactgactctcggtgtgctgttcggcaaagtctttagtcagaccacaatatgtcgattcgaagcccttcaactgtcatttaagaatatgtgcaaacttcgacctctgctccagaaatgggtcgaagaggcggataataacgagaacctgcaagaaatctgtaaggcggagactctggttcaagctcgcaaaaggaaacgtacgtctatagaaaatagagtccgtgggaatcttgaaaacctgtttctccaatgtccaaagcctactttgcaacaaatatctcatattgcgcaacaactcggcctggaaaaggacgtagttagagtctggttttgcaatcgcagacagaaagggaaacggtcttccagtgattacgcgcagagggaagacttcgaagcagccggttcaccgttttccggcggcccggtatctttcccattggctcccggtcctcacttcggcacacccgggtacggctcaccacattttaccgccctttattcaagcgttccctttccggaaggcgaggctttcccgccggtgtcagtgactacacttggatccccaatgcacagcaattag

SEQ ID NO: 2 - DNA, 954 bp, synthetic construct
  source 1..954: mol_type = other DNA
atgtataatatgatggaaaccgaattgaaaccacccgggccccaacagacctctggcggcggtggtggtaatagcacagcagcagccgctggtggaaatcaaaagaattctccagatagagtgaaacgacctatgaacgcatttatggtctggtctagaggacaacgaaggaaaatggctcaagaaaatcccaaaatgcataatagcgaaatttccaaacggttgggtgcggaatggaagctcctcagcgaaaccgaaaagaggccatttattgatgaagcgaaaagactcagggcattgcatatgaaagaacatccagactacaagtatagaccacgccgcaagacaaagactctgatgaagaaggacaaatataccctgcctggtggattgttggctcctggcggtaacagtatggcttctggcgtgggcgttggggctggacttggtgccggggtcaatcaacgaatggattcctatgcccatatgaatggatggagtaatggttcctattctatgatgcaagatcaattgggatatcctcaacatcccggtctgaacgctcatggtgctgctcaaatgcaacctatgcatcggtatgatgtaagtgcattgcaatataatagcatgacatccagtcaaacatatatgaatgggtcaccaacatatagtatgagctattcccaacaaggtacaccagggatggccctggggagcatggggagtgtcgttaaaagtgaagcttcaagttcaccacccgtagtgacgagttcttcacattctcgagccccatgtcaagcaggagatcttagggatatgatttcaatgtacttgccaggggctgaagtccccgagccggcagcgccttctaggctgcatatgtctcaacattatcaatccggacccgttccagggaccgctatcaatggtacgttgccattgtcccatatgtaa

SEQ ID NO: 3 - DNA, 1365 bp, synthetic construct
  source 1..1365: mol_type = other DNA
atggacttctttcgagtggtagagaatcaacaacccccggccaccatgccattgaatgtatcatttacaaaccggaactacgacctggactatgattcagttcagccttacttttattgtgacgaagaagaaaatttctatcaacaacaacaacaatcagaattgcaaccacccgctccatcagaagacatttggaagaagtttgaactcctgccaactccaccgctcagcccgagtagacgttctggactgtgttctccttcttatgtggctgtgactccgttttcactgcgtggcgataatgatggcggcggtggctcttttagtacagcagatcaacttgaaatggtcacagaactccttggtggtgatatggttaatcaatcattcatttgtgatcccgatgatgagacatttataaagaacatcatcatacaagactgcatgtggtcagggtttagtgctgctgcgaaactggtgagcgaaaagttggcttcttatcaagccgcccggaaggatagtggatcaccaaatccagcaaggggtcattcagtgtgtagcacaagctctctgtatcttcaagacctctccgcggctgcaagtgaatgtattgatccaagtgtcgttttcccttatcccctgaatgattcttcctctcctaaaagctgtgcgagccaggattcttcagctttctccccaagctccgacagtttgttgagctctactgaaagtagtcctcaagggtcaccggaacctctcgtccttcacgaagaaacaccccctacaactagttccgattccgaagaagaacaggaagacgaagaggagattgacgtggtatcagttgagaaaagacaagcccccgggaagcgaagcgaaagcgggagcccaagcgccggcggacattccaagcccccacattctcctttggtactgaaaagatgtcatgtgagcacccaccaacataattatgctgctcccccatcaaccaggaaagattaccccgccgctaaacgagttaaactggattcagtgagggttcttaggcaaatttcaaataataggaagtgtacttcacctcgcagtagcgatacagaagaaaacgttaaaagacggacgcataatgtgctggaacgacaaagacgaaatgaacttaagaggtccttcttcgcgcttagggatcaaatacctgaactggagaataacgagaaagctccaaaagtggtgattttgaagaaggctactgcgtatatactttctgtacaggccgaagaacagaaactgatatcagaggaagatctgcttcgtaagcgcagagagcaactgaagcataagctcgagcaactccgcaatagctgcgcctag

SEQ ID NO: 4 - DNA, 1542 bp, synthetic construct
  source 1..1542: mol_type = other DNA
atgcggcagccaccaggggaaagtgatatggccgtttccgacgctcttctgccttcattttcaacctttgcttccggacctgccgggcgggaaaagacgcttaggcaggccggagcacccaacaatcgatggagagaagaactgagccatatgaaaagactgccgcctgtactcccggggcggccatacgatctcgccgccgctacagtagcaactgatttggaatccggtggtgctggggcagcctgtggcggatctaatcttgctcctctgccaagacgggaaacggaagaatttaatgacctgctcgatttggatttcatcctttctaactcacttacacacccaccagaaagtgtcgctgctacggtctctagctctgccagcgcaagcagttcatcttctccatcttcctcaggacccgcaagtgccccgagcacttgttcctttacgtacccaatacgagctggcaatgatcctggagttgctcccggtggtacaggcggtggtctgttgtacggaagagaatcagccccaccaccaaccgcaccatttaatttggccgatattaatgatgtttcacctagtggtggttttgttgcggaactgctccgtcccgagctggatcccgtctatatcccaccacaacaaccccaaccacctggcggcggattgatgggtaaatttgttcttaaagcatccctttccgcaccagggtcagaatatggaagtccctcagtgatttccgtatctaagggatcccccgatgggtcccatccagttgtcgttgccccttataatggtggcccgcctagaacctgtccgaaaataaaacaagaagctgtatcatcatgtacgcatctcggggcaggtccacccctgtctaacgggcatcgccctgcagcccatgattttcctttgggccgacaactgccgtcccgtacaactccaacactcggcctcgaagaggtcctcagtagtagagattgccatccagcactccccctgccaccaggttttcaccctcatcccggtcctaactatcccagctttctcccagaccaaatgcaaccccaggttcccccacttcactatcagggccaaagcagaggtttcgtggcgcgagccggagaaccatgcgtctgttggcctcattttggcacccatggaatgatgttgacacccccaagtagtccactcgaactgatgccccctggcagttgtatgcctgaagaacctaaacccaaaagagggcggcggagttggccacgaaagcgaacagcaactcatacctgcgactatgctggatgtgggaagacatataccaaatctagccacctgaaagcgcatctccgcacgcatactggagaaaagccctatcattgcgattgggatgggtgcggctggaagtttgcacgatctgacgagcttactagacattatcgcaagcataccggacatcggccctttcaatgtcagaagtgtgatcgcgccttcagccggagcgatcatctggctctgcatatgaaacgacacttctag

SEQ ID NO: 5 - DNA, 630 bp, synthetic construct
  source 1..630: mol_type = other DNA
atgggctcagtcagcaatcaacaattcgcgggtggatgtgcaaaagctgctgaggaagccccagaagaagctccagaagatgccgctcgcgccgctgatgaaccacaactgctccatggagctgggatttgcaaatggtttaatgtccggatgggctttggtttcttgtctatgacagcaagagctggagtggccctcgatccacccgtcgacgtgttcgtacatcaatctaaacttcatatggaaggatttcgctcactcaaagaaggagaagccgtagaatttacattcaagaaaagtgcgaaagggcttgagagcatacgggtgacagggccaggcggcgtgttttgcatcggatcagaacgaaggcccaagggtaaatccatgcaaaagaggcgatccaaaggggatcggtgttataattgcggtggccttgaccaccacgcgaaagagtgtaaacttcccccgcaacctaagaaatgtcatttctgtcaatccatatcacacatggtggcgagctgcccattgaaagcacaacaaggaccgtccgcccaagggaaacctacttatttcagggaagaggaagaggagattcatagtcccactctccttcctgaagcccaaaactag

SEQ ID NO: 6 - DNA, 918 bp, synthetic construct
  source 1..918: mol_type = other DNA
atgtcagtcgacccggcctgcccacagagtctgccctgtttcgaggcttcagattgcaaggagtcctctccgatgcccgtcatctgcggacccgaggagaattaccccagtctgcagatgtccagtgccgaaatgccacatacagaaacggtttcaccgctcccatcttcaatggaccttttgatccaagattctcccgacagcagcacttccccaaagggaaagcagcctacctcagcggagaaatctgtggccaagaaagaggataaagttcctgttaagaagcaaaagacaaggaccgtattttcctcaacacaactctgcgtccttaacgaccggttccaacgccaaaagtatctgtccttgcaacaaatgcaggagctgagcaatattttgaatctgtcttataagcaagtcaagacttggtttcaaaaccagcgtatgaagagtaaaagatggcagaagaataattggcccaagaactctaacggcgttactcaaaaggcgagtgcccccacttatccatctctgtattcatcatatcatcaaggctgtttggttaatcccacgggcaatctgccgatgtggtccaatcaaacgtggaataacagtacttggtccaatcaaactcaaaatatacaaagttggtcaaatcattcttggaatacccaaacgtggtgtactcagagctggaataaccaagcatggaactccccattttacaattgcggcgaagagtcactccaaagctgtatgcaatttcaacccaacagtcccgcatccgatcttgaagcagcactggaagcggccggtgaggggctgaacgtgattcaacaaacaacaagatacttctccacacctcagactatggaccttttcctgaattattcaatgaatatgcagcccgaggatgtctag

SEQ ID NO: 7 - DNA, 115 bp, synthetic construct
  source 1..115: mol_type = other DNA
aaaaaaaaaaaaaaaaaaaaaaaaaaaaaaaaaaaaaaaaaaaaaaaaaaaagcatagcagctgactaaaaaaaaaaaaaaaaaaaaaaaaaaaaaaaaaaaaaaaaaaaaaaaa

SEQ ID NO: 8 - DNA, 87 bp, synthetic construct
  source 1..87: mol_type = other DNA
ttggaccctcgtacagaagctaatacgactcactatagggaaataagagagaaaagaagagtaagaagaaatataagagccaccatg

SEQ ID NO: 9 - DNA, 119 bp, synthetic construct
  source 1..119: mol_type = other DNA
gctgccttctgcggggcttgccttctggccatgcccttcttctctcccttgcacctgtacctcttggtctttgaataaagcctgagtaggaagtgagggtctagaactagtgtcgacgc

SEQ ID NO: 10 - DNA, 1863 bp, synthetic construct
  source 1..1863: mol_type = other DNA
atggccgaagccagaacatctctgtctgcccactgtagaggccccctggctacaggtctgcaccccgacctggatctgcctggccggagcttggccacccctgctcctagctgctacctgctgggctctgaacccagctccggccttggcctgcagcctgagacacacctgcctgaaggctccctcaagcggtgctgcgtgctgggcctgcctccaacatcccctgcttcctcttctccatgcgccagcagcgacgtgaccagcatcatcagaagcagccagacaagcctcgtgacctgcgtgaacggcctgcggagccctcccctgaccggcgatctgggaggcccctcaaagagagcccggcccggccctgccagcaccgacagccacgagggcagcctgcagctggaagcttgtagaaaggccagcttcctgaaacaggagcctgccgacgagttctccgagctgttcggcccccaccagcagggactgccgcctccttatcccctgtcccagctgcccccaggaccaagtctgggaggactcggcctgggactggccggcagagtagtggccggaagacaggcctgccggtgggtggactgttgtgccgcttacgagcagcaggaggaactggtgcgccacatcgagaaaagccacatcgaccagaggaagggcgaggatttcacatgcttctgggccggctgcgtgcggcggtacaagccctttaatgccagatacaagctgctgatccacatgagagtgcacagcggcgaaaagcctaacaagtgcatgttcgagggctgcagcaaggccttctctagactggaaaatctgaaaatccacctgagatctcacaccggagaaaagccttacctgtgccagcatcctggatgtcagaaggccttcagcaacagctctgatagagccaagcaccagagaacccacctggacaccaagccttatgcctgccagatccctggctgcagcaaaagatacaccgatccttctagcctgcggaagcacgtgaaggcccatagcgctaaggaacagcaggtgcgcaaaaagctgcatgccggcccagataccgaggccgatgtgctaactgagtgcctggtcctgcaacagctgcacacatctacccagctggccgccagcgacggcaagggcggatgtggcctgggacaggagctgctgcctggtgtttaccccggaagcatcacccctcacaacggcctggccagcggactgctgccccccgcacacgacgtgcctagccggcatcaccctctggacgccaccaccagcagccaccaccacctgagccccctgcctatggccgaaagcaccagagatggcctcggccctggcctcctcagccctatcgtgtctccactgaaaggcctgggcccccctcctcttcctccaagcagccagagccacagccccggcggccagcctttccctaccctgccttctaagcccagctaccctccttttcagagccctcctccccctccactgccctctcctcagggctaccaaggcagcttccactctatccagtcctgttttccttacggcgactgctacagaatggccgaaccagccgccggcggcgacggcctggtgggcgagacacacggctttaaccccctgagacctaacggctaccactctctgagcacacctctgcctgctaccggctacgaggctctggcagaggccagctgccctaccgccctgccgcaacaacctagcgaagacgtcgtgtcctctggcccagaggactgcggattcttccccaacggcgccttcgaccactgcctgggccacattcctagcatctacaccgacacatga

SEQ ID NO: 11 - DNA, 1281 bp, synthetic construct
  source 1..1281: mol_type = other DNA
atggagttcgccatggagctgctgtcgccaccgttgcgtgacgtggacctgacagcccccgacggctctctgtgctcctttgctactaccgacgatttctacgatgacccgtgctttgattctcccgacctgcgctttttcgaagacttagatccgcgcctgatgcatgtaggtgctctgctaaagcccgaggagcacatggctggccacttggcttccgacttcgcgttctccccgccacccggcggcggaggcgatggccctggcggacccgagccaggttgggtcgaccctcggacctggctctccttccagggccccccaggcgggcccggcataggtcccggcgttggacccgggagcgaggtgtggggcatccctccgtgcccacccccgtatgagttctgcgggggtatggcctactgcgggcctcaggtgggcgtcggcctcgtccctcaggggggtctggagacctctcagccggagggagaggctggggtcggcgtggagagcaactccgacggggcgtcgcccgaaccttgcactgtcacgcccggggccgttaagctggagaaggagaaacttgagcagaaccccgaggagagccaggacatcaaggcgctgcagaaggagctggaacagttcgccaagctgctgaagcagaagcgcatcaccctaggttacacccaggcggacgtgggcctgacgcttggtgtgctgttcggaaaggtgttcagccagacgaccatctgccgattcgaggccctccagctgtccttcaagaacatgtgcaagttgcggcccctgctccaaaaatgggtggaggaggctgacaacaacgagaatctccaggagatctgtaaagccgagactctggtgcaggcccgcaaacgcaagcgtacctcgattgaaaacagggtgcgtggcaacctggagaacctgttcctacagtgtcccaagcctacccttcagcagattagccacatcgcacaacagttgggccttgaaaaggatgtggtacgcgtgtggttctgtaaccgccgccagaagggtaagcgcagctccagcgactacgcgcagagagaggactttgaggctgcaggatctcctttttctggcggccctgtgagtttccctctggccccgggaccccactttggtactccgggctacggctccccgcacttcaccgccctgtactctagtgtcccgtttcccgagggcgaggcgttccccccagtgtccgtgaccacactggggtccccaatgcattcaaattga

SEQ ID NO: 12 - DNA, 1056 bp, synthetic construct
  source 1..1056: mol_type = other DNA
atgacaatgaaaatgatggtgcacatctacttcgtctctcttctgctgctgctgtttcacagctacgcaattgacatcgaaaacgagatcaccgagttcttcaacaagatgcgtgacacccttcccgccaaggattctaaatggctcaacccggcctgcatgtttggaggcaccatgaacgacatcgcggcgctgggcgagcccttctccgctaaatgtcccccgatcgaagattctctgctgagccaccgctacaaggactatgtggtcaaatgggagcgcctggagaagaaccgtcggcgccaggtctcaaacaagcgcgtaaagcatggagatctttggatcgccaactacaccagtaaatttagcaaccgccgctacctgtgcactgtcaccaccaagaatggggactgcgtgcagggcatcgtgcggagccacatccgcaagcctccctcttgtattcccaagacctacgagctggggacacatgacaagtacggcattgacctgtattgcgggatcctgtacgcgaagcactacaacaacatcacctggtacaaggacaacaaggagattaacatcgatgacatcaagtactcccagactggcaaggagctcatcatccacaaccctgagctggaggactccggtagatatgattgttacgtgcattacgacgatgtgcgcatcaaaaacgacattgtcgtttccaggtgtaagatactcactgtgatccctagtcaggaccaccgcttcaagctgatcttggacccgaagataaatgtgaccatcggggagccagccaatatcacctgcactgccgtgtccacctcgttgctcattgacgatgtactgattgagtgggagaatccatcgggttggctaatcggcttcgacttcgacgtgtactcggtgctaacctcccgtggtggcatcactgaggctacgctttatttcgagaacgtgacggaggagtacatcggcaacacctacaagtgccgaggccacaactactacttcgaaaaaaccctgacgacgaccgtggttttggagtga

SEQ ID NO: 13 - DNA, 1542 bp, synthetic construct
  source 1..1542: mol_type = other DNA
atgcgtcagccacccggggagagcgacatggccgtgtcggacgcgctgctgccatccttttccaccttcgcctcgggtccggccggccgagagaagactctgcgccaggccggagcccctaacaaccgctggagagaggagctgtcacacatgaaacgcctgccccccgtgctgcctgggcgcccctacgaccttgccgcggccacggtggctaccgacttggagtctggaggtgctggagcagcgtgtggcggaagcaacctggcaccgttgccacgccgggagaccgaggagttcaacgacttgttggatctggactttattctgtccaactcccttacacacccgcccgagagcgtagcagccaccgtgagctccagtgcttccgcttcctcctcatccagcccgtcgtcttctggccctgcctctgcgccgtcgacctgttcgttcacctatcccatccgggccggcaacgatccgggcgtggccccgggcggcaccggtggtggtctcctgtacggcagggagtccgccccccctccaaccgctcccttcaacctcgcggacatcaatgacgtgtccccctctggcggcttcgttgcagaactgttaaggcctgaactggatccagtgtacatcccgccccagcagcctcagccgccgggcggcggtctgatgggcaaatttgtcctgaaggcgtctctgtctgctcctggctccgagtacggcagccccagtgtgattagcgtgtctaaaggcagccccgacgggtcgcaccccgtggtggtcgctccttacaacggtggacccccgcgcacctgcccaaagatcaagcaggaggctgtttcttcatgcactcatctaggcgccggtccacccctttccaatggccaccggcccgccgcgcatgacttccccctgggccgccagctgcccagccggaccacacctaccttaggcctggaggaggtgcttagttcgcgcgactgtcatcctgccctgcctctccctccaggcttccacccccacccgggacccaactacccgtccttcctgcccgaccagatgcaacctcaggtccctcccctgcactaccagggacagagccgcggtttcgtcgcccgtgctggcgagccatgcgtctgttggccgcacttcggcacgcatggaatgatgctaactcccccgagctcccccctggagctgatgcccccgggttcctgtatgccagaggagcccaagcctaagcgcggcagacgtagttggccccgtaagcgcaccgccacacatacgtgcgactacgcggggtgcgggaaaacctacaccaagtcttctcacctcaaggctcacttgcgtactcacactggagagaagccttatcactgcgactgggatgggtgcgggtggaagtttgctcgctccgacgaactcacccgccattaccgcaagcacaccggccaccgccccttccagtgtcagaagtgcgatcgagcgttctcccgctcggaccacctggccctacacatgaagaggcacttttga

SEQ ID NO: 14 - DNA, 630 bp, synthetic construct
  source 1..630: mol_type = other DNA
atgggctccgtgtcgaatcagcagttcgcaggcgggtgcgccaaggcagccgaggaggccccggaggaggctcctgaagacgccgctcgcgcggcggacgagcctcagctactccatggagctggcatctgcaaatggttcaacgtgcgaatgggatttggcttcctgtctatgactgctcgtgccggcgtggcgctggacccgcccgtagacgtgttcgtccaccagagcaagctgcatatggagggtttcagatctctgaaggagggcgaggccgtggagttcacgttcaagaagtcggccaaaggtctggagagcatccgcgtcaccggtcccggcggcgttttttgtattggctccgagcgccggcccaagggcaagtccatgcagaagcgccgctccaagggggacaggtgttacaactgcgggggtttggatcaccacgcgaaggagtgcaaacttcctcctcagccgaagaaatgtcacttttgtcagagcatcagtcacatggtggcttcatgcccactcaaggcccaacagggaccctctgcgcagggcaagcccacctacttccgcgaggaggaagaagagatccactccccaaccctgctgcccgaggctcagaactga

SEQ ID NO: 15 - DNA, 918 bp, synthetic construct
  source 1..918: mol_type = other DNA
atgtctgttgacccggcctgcccacagagccttccctgctttgaggcttccgactgtaaagagagttccccgatgcccgtgatttgtggtcctgaagagaactacccgtccctacagatgtcatcggcggagatgcctcataccgagaccgtgtcccccttaccctcttctatggatctgctgatccaggactcgcccgacagctcgaccagccctaagggcaagcagccaacctccgcagagaagtccgtggctaagaaggaggacaaagtaccggtgaagaaacagaagactcgcaccgtgttctcgtcgacccagctgtgcgtgctcaacgacaggtttcagcgccagaagtacctgtccctgcagcagatgcaagagctcagcaacatcctgaacctgagctacaagcaggtcaagacctggttccagaaccagcgtatgaagtccaagcggtggcaaaaaaacaactggcccaagaacagcaacggtgtcacccagaaggcctccgcccccacctacccgtctctttactccagctatcaccagggctgcctggtgaatccaacaggaaacctgcccatgtggtctaatcagacctggaacaactccacctggtctaaccagacacagaacatccagagttggtctaatcactcttggaacacgcagacctggtgcactcagagttggaataaccaggcctggaattcccccttctacaactgcggggaggagagcttgcagtcatgtatgcagttccagcccaactccccagccagtgacctggaggccgcgctggaggcggctggcgagggcctcaacgtgatccaacagacgacccgctacttctccacgcctcagactatggatttgttcctgaactattcgatgaacatgcagcctgaagatgtgtga

SEQ ID NO: 16 - DNA, 1083 bp, synthetic construct
  source 1..1083: mol_type = other DNA
atggcgggccacttggcttccgacttcgcgttctctcccccgcctggcggaggcggggacggccccggcggccctgagccaggctgggtcgatccacgcacctggctctccttccagggaccccccggggggcccggcatcggtcccggggtaggccccggatcggaggtgtggggcatcccgccgtgcccaccgccctatgagttctgtgggggtatggcctactgcgggcctcaggtcggtgtcggtctggtgcctcagggcggtctcgagaccagccagccggagggcgaggctggcgtgggtgtggagagcaacagcgatggcgcttctcctgaaccatgcactgtcacccccggcgccgtgaagctggagaaagagaaactggagcagaatccagaggagagtcaggacatcaaggccctgcagaaggaactggaacagttcgccaagctgctgaagcagaagcgtatcacccttggatacacccaggcggacgtgggcctcactttaggcgttcttttcggaaaggtgttctcacagaccacaatctgccgattcgaggccctgcaactttctttcaaaaacatgtgcaagttgcggcccctactgcagaaatgggtggaggaagcggacaacaacgagaatctgcaggagatctgtaaggccgagacactggtgcaggctcgcaagcgcaaaagaacgagcattgagaaccgcgtccgcggcaacctggagaacctgttcctgcagtgcccaaagcctaccctccagcaaattagccacatcgctcagcagctgggcctggagaaggatgtggtgagggtgtggttctgtaaccggcgccagaagggcaagcgcagctccagtgactacgcacagcgtgaggactttgaagccgccggctccccgttttcaggaggccccgtgtcctttcctttggctcccggccctcatttcggtactccgggctacggctccccccactttaccgccctttactcctctgtccccttccccgagggagaggcatttcccccggtgtccgtaacgaccctgggttccccaatgcactctaactga

SEQ ID NO: 17 - DNA, 954 bp, synthetic construct
  source 1..954: mol_type = other DNA
atgtataacatgatggaaacagagctgaagcccccggggcctcaacagacctccggcggtgggggcggcaactcgaccgctgccgcagctggtggaaaccagaagaacagtcccgacagagttaagcgcccgatgaacgcgttcatggtgtggtctcgcggccagcgccgcaagatggcgcaggagaatccaaaaatgcacaactcggagatctccaagcggctcggtgccgagtggaagctgctaagcgagaccgagaaacgtccttttattgacgaggccaagcgcctgcgtgcgcttcacatgaaggagcaccccgactacaagtacaggccccgacgcaaaaccaagaccctgatgaaaaaggacaagtacaccctccccggcggcctgctggcccctggtggcaacagcatggcctccggagtcggggtaggcgccggccttggagctggagtcaaccagcgtatggattcttacgcgcacatgaatgggtggtcaaatggctcgtattctatgatgcaggaccagctgggctaccctcaacaccccggcctcaacgcccatggagcggctcagatgcagccaatgcaccgctacgatgtgagcgccctgcagtacaactctatgactagttcacagacttacatgaacggttccccaacctactccatgtcttacagtcagcagggaacgccgggtatggctctgggctccatgggctccgtggtgaagtcggaggcatcctccagccctcccgtggtcacctcctcctctcacagccgcgctccttgccaggccggggacctgcgcgacatgatctctatgtatctgcccggtgcagaggtgcctgaaccggcggccccctctcggttgcatatgtcccagcattaccagagcggcccggtgccaggcactgccatcaacggcaccttgcccctgagccacatgtga

SEQ ID NO: 18 - DNA, 1365 bp, synthetic construct
  source 1..1365: mol_type = other DNA
atggatttcttccgagtggtggagaatcagcagccgcctgccaccatgccccttaacgtgtccttcactaacagaaactacgacctggactacgacagtgtccagccctatttctactgtgatgaggaggagaacttttaccagcagcaacagcagagcgaactgcagcccccagcgccctcggaggacatctggaagaaatttgagctgctgccaaccccccccctatctccatcccgccgttccggtctctgctctcctagctacgtggctgtcactcccttttcgctgcgtggggacaacgatggggggggcggaagtttcagcactgcggaccagctggagatggtgaccgagctgctgggtggtgacatggtcaaccagtcttttatctgcgacccggatgacgagaccttcatcaagaacatcatcatccaggactgtatgtggtctggcttctctgccgctgcaaaactggtctcggagaaacttgctagctaccaggctgctcgcaaggactccggctcgccgaatccagctaggggacatagtgtttgtagtacctcctcgctctacctgcaggacctgtccgcagcggcctctgagtgtattgacccgtccgtggtgttcccctatcctctcaacgactcgtcttcccccaagagctgcgcctcccaggactcatctgcgttctccccctcctccgatagcctgttgagtagcacagagagctcccctcagggctccccggagcccctggtgctacacgaggagaccccgcccaccaccagcagtgactcagaagaggagcaggaggacgaagaggagatcgatgtggtttccgtggagaagcgccaggcccctggcaaacgctccgaatccggctccccttccgccggcggccactccaagcccccgcacagcccgttggtgctgaagagatgtcacgtgtcaacccaccagcataactacgcggcgcctccctctacccgcaaagactaccccgctgccaagcgcgtgaagttggatagcgtccgggtcttacgccagatttctaacaaccgcaagtgcacttctccaaggtcttccgacacggaggagaacgtgaagcggcgcacccacaacgtactggagcgccagcgtcgtaacgagttgaagcgcagcttcttcgcgcttcgggaccagatacctgagctcgagaataacgagaaagcaccaaaggtagtcatcctgaagaaggccacggcctacatcttatcagtgcaggccgaggagcagaagctgatttcagaggaggatctgctgcgcaagcgccgagaacagctgaagcacaagctggaacaactccgcaactcatgcgcctga

SEQ ID NO: 19 - DNA, 1863 bp, synthetic construct
  source 1..1863: mol_type = other DNA
atggccgaggcccgcactagcctctcagcgcactgtcgcggccctctcgctacgggcctgcaccccgacctggacctgcccgggcgcagcctggctaccccggccccctcctgttaccttttgggctcggagccctctagcggcctgggcctacagccagagactcaccttcccgaaggctccctgaagaggtgttgcgtccttggcttgccgcccacctcgcctgccagctcctccccatgcgcgtcttcagacgtgacctccatcatccgctcgtcccagacctccctggtgacctgcgtgaacgggctgcgttctcccccgctcaccggcgacctgggaggtccgagtaagcgcgctcggcccggccctgcgagtacagactctcatgagggctcgctgcagctggaagcgtgtcgcaaggcctccttcctgaagcaggagccagccgacgagttctctgagctgttcggacctcaccagcagggtttgccccctccttacccactcagccagctcccccctgggccctctttgggcggtttaggcctggggttagcgggacgtgtggtggctggccgccaggcctgccgatgggtcgattgttgtgccgcttacgagcagcaggaggagctggtgcgccacattgaaaagagccatattgaccagcgcaagggcgaggacttcacctgcttttgggctgggtgcgtgcgccgctacaagccgttcaacgcgagatacaagctgctgatccacatgcgtgtacactccggagagaaacccaacaagtgcatgtttgagggatgctccaaggcgttcagccgcctggagaacttgaaaatccacctgcgctcacacaccggtgagaagccatacctgtgccaacaccccggttgtcagaaggcgttcagcaacagctccgatcgagctaaacaccagcggacccatcttgacaccaagccgtacgcatgccaaattccagggtgctccaagcgttacaccgacccgtccagtctgcgcaagcacgtgaaggcccatagtgcgaaggagcagcaagtgcggaagaagctccacgccggccctgacacggaggccgatgtgctgaccgagtgcctggttctgcagcagctgcacactagcacccagctggcagccagcgatggcaaaggtggttgcgggctgggacaggagttactgccaggcgtgtaccccggaagcatcaccccgcacaacggcctagcctctggacttctccctcctgcccatgacgtgccctctcggcaccacccgctcgatgctaccacctcctctcaccaccacctgtcccccttgcctatggccgagtccacccgcgacggcctgggccctggcttgttgtcgcccatcgtgtcccctctgaaaggccttggccccccgccgctgcccccctcttctcagtcccatagtcccggtgggcagccgtttcccaccctgccctctaagccctcctatcccccgttccagagtccacccccccccccactgccgtccccgcagggctaccagggctccttccattctatccagtcttgctttccttacggcgattgttacaggatggctgagcccgcagctgggggcgatgggctggtgggcgagacacatggattcaatccactcaggcccaacggttatcactcgctatccactccgttgcctgccacgggttatgaggcgctggcagaggctagctgcccaacggctctgcctcagcagccatcagaggacgtggtcagcagcggccctgaagactgcggcttctttcctaatggggccttcgaccactgtcttggccacatcccgtccatctacacagacacctga

(19) United States
(12) Patent Application Publication
(10) Pub. No.: US 2023/0042860 A1
(43) Pub. Date: Feb. 9, 2023

(54) POLYCISTRONIC EXPRESSION VECTORS

(71) Applicant: TURN BIOTECHNOLOGIES, INC., Mountain View, CA (US)

(72) Inventors: Naveen BOJJIREDDY, Milpitas, CA (US); Tapash Jay SARKAR, Tomball, TX (US)

(21) Appl. No.: 17/812,710

(22) Filed: Jul. 14, 2022

Related U.S. Application Data
(60) Provisional application No. 63/222,297, filed on Jul. 15, 2021.

(51) Int. Cl. C12N 15/85; C12N 5/074

(52) U.S. Cl. CPC C12N 15/85; C12N 5/0696; C12N 2501/60

(57) ABSTRACT

The subject matter described herein relates to compositions and methods for cellular rejuvenation, tissue engineering, and regenerative medicine by transient exposure of cells or tissues to synthetic, non-integrative mRNAs encoding reprogramming factors. Reprogramming factor encoding polynucleotides and corresponding polypeptides that trigger less immune response, are more stable, and/or exhibit altered activity than wild-type reprogramming factors are provided. RNA vectors expressing one or more of the improved reprogramming factor polynucleotide sequences are also provided.

### CROSS-REFERENCE TO RELATED APPLICATIONS

This application claims the benefit of U.S. Provisional Application No. 63/222,297, filed Jul. 15, 2021, which is incorporated by reference herein.

### REFERENCE TO SEQUENCE LISTING, TABLE OR COMPUTER PROGRAM

A “Sequence Listing is submitted with this application in the form of a text file, created Jul. 11, 2022, and named “111277-0041-8001US00_SEQ.xml” (35 kilobytes), the contents of which are incorporated herein by reference in their entirety. Peptide sequences related to the present disclosure are also provided in Table 1.

### TECHNICAL FIELD

The subject matter described herein relates to compositions and methods for cellular rejuvenation, tissue engineering, and regenerative medicine by transient exposure of cells or tissues to synthetic, non-integrative mRNAs encoding reprogramming factors.

### BACKGROUND

Aging is characterized by a gradual loss of function occurring at the molecular, cellular, tissue and organismal levels. At the chromatin level, aging is associated with the progressive accumulation of epigenetic errors that eventually lead to aberrant gene regulation, stem cell exhaustion, senescence, and deregulated cell/tissue homeostasis. The technology of nuclear reprogramming to pluripotency, through over-expression of a small number of transcription factors, can revert both the age and the identity of any cell to that of an embryonic cell by driving epigenetic reprogramming. The undesirable erasure of cell identity is problematical for the development of rejuvenative therapies because of the resulting destruction of the structure, function and cell type distribution in tissues and organs. There is a need for methods of rejuvenating cells that avoid dedifferentiation and loss of cell identity and that provide convenient and simple treatment paradigms. The present disclosure addresses this need, and provides additional benefits as well.

A major cause of aging is now thought to be due to epigenetic changes that cause cells to transcribe the wrong genes at the wrong time for optimal function, a process that becomes more dysfunctional over time, leading to diseases, an inability to heal and eventually to death. The Yamanaka factors (OCT4, SOX2, c-Myc, and KLF4) have previously been shown to induce pluripotency in vitro (Takahashi et al., Cell. 2006 Aug. 25; 126(4):663-76) and reverse the DNA methylation clock of aging (Horvath, Genome Biol. 2013). Nanog and Lin28 can help induce pluripotency together with Yamanaka factors. And Ted, NR5A-2, Sall4, NKX3-1 can replace Oct4 (Gao et al., Cell Stem Cell 12, 1-17, Apr. 4, 2013 and Mai et al., Nature Cell Biology 20, 900-908, 2018).

### BRIEF SUMMARY

The following aspects and embodiments thereof described and illustrated below are meant to be exemplary and illustrative, not limiting in scope.

In one aspect, a reprogramming factor encoding polynucleotide having at least 95% sequence identity to any one of SEQ ID NOs: 1-6 or 10 is provided.

In another aspect, a reprogramming factor protein or polypeptide encoded by a polynucleotide having at least 95% sequence identity to any one of SEQ ID NOs: 1-6 or 10 is provided.

In another aspect, an RNA vector, comprising one or more reprogramming factor polynucleotide sequences, wherein the one or more polynucleotide sequences comprises at least 95% sequence identity to any one of SEQ ID NOs: 1-6 or 10. In some embodiments, the RNA vectors provided herein include one or more reprogramming polynucleotide sequences that encode a reprogramming protein or polypeptide with altered secondary or tertiary structure compared to wild-type reprogramming factor proteins or polypeptides. In some embodiments, the RNA vectors provided herein produce reprogramming proteins or polypeptides that trigger a reduced immune response, or exhibit altered activity or stability, compared to wild-type reprogramming factor proteins or polypeptides.

In some embodiments, the RNA vectors described herein comprise, from 5′ to 3′: (a promoter)-(a first polynucleotide sequence for a first reprogramming factor)-(a first reprogramming factor separating region)-(a second polynucleotide sequence for a second reprogramming factor)-(a second reprogramming factor separating region)-(a third polynucleotide sequence for a third reprogramming factor)-(optional additional polynucleotide sequences for optional additional reprogramming factors)-(optional additional separating regions)-(optional selectable marker)-(virus 3′UTR or a tail)-(optional selectable marker)-(optional promoter). In some embodiments, the reprogramming factor separating regions each consist of one or more of the following: an IRES, a promoter and a self-cleaving peptide.

In some embodiments, the RNA vectors provided herein, comprise a reprogramming factor such as Oct, Sox, Klf, Lin, Nanog, Myc or Glis. In some embodiments, the reprogramming factor is OCT4, SOX2, KLF4, LIN28, NANOG, c-Myc, or Glis1.

In some embodiments, the reprogramming factor comprises OCT4, wherein the OCT4 consists of a nucleotide sequence having at least 95% sequence identity to SEQ ID NO: 1. In some embodiments, the reprogramming factor comprises SOX2, wherein the SOX2 consists of a nucleotide sequence having at least 95% sequence identity to SEQ ID NO: 2. In some embodiments, the reprogramming factor comprises c-Myc, wherein the c-Myc consists of a nucleotide sequence having at least 95% sequence identity to SEQ ID NO: 3. In some embodiments, the reprogramming factor comprises KLF4, wherein the KLF4 consists of a nucleotide sequence having at least 95% sequence identity to SEQ ID NO: 4. In some embodiments, the reprogramming factor comprises LIN28, wherein the LIN28 consists of a nucleotide sequence having at least 95% sequence identity to SEQ ID NO: 1. In some embodiments, the reprogramming factor comprises NANOG, wherein the NANOG consists of a nucleotide sequence having at least 95% sequence identity to SEQ ID NO: 6. In some embodiments, the reprogramming factor comprises GLIS1, wherein the GLIS1 consists of a nucleotide sequence having at least 95% sequence identity to SEQ ID NO: 10.

In some embodiments, the present technology provides an RNA vector wherein the first polynucleotide sequence comprises an OCT4 nucleotide sequence having at least 95% sequence identity to SEQ ID NO: 1, the second polynucleotide sequence comprises an SOX2 nucleotide sequence having at least 95% sequence identity to SEQ ID NO: 2, and the third polynucleotide sequence comprises an KLF4 nucleotide sequence having at least 95% sequence identity to SEQ ID NO: 4.

In some embodiments, the present technology provides an RNA vector wherein the first polynucleotide sequence comprises an LIN28 sequence having at least 95% sequence identity to SEQ ID NO: 5, the second polynucleotide sequence comprises an NANOG sequence having at least 95% sequence identity to SEQ ID NO: 6, and the third polynucleotide sequence comprises an c-Myc sequence having at least 95% sequence identity to SEQ ID NO: 3.

In some embodiments, the present technology provides RNA vectors wherein the first polynucleotide sequence, second polynucleotide sequence and third polynucleotide sequence are independently selected from the group consisting of nucleotides comprising at least 95% sequence identity to SEQ ID NOs: 1-6 or 10.

In some embodiments, the RNA vectors provided herein are transcription vectors comprising a transcription initiation region. In some embodiments, the RNA vectors include a poly A tail. In other embodiments, the RNA vectors include tails that comprise a heteropolymer insert, such as a tail having at least about 80%, about 85%, about 90%, about 95%, about 96%, about 97%, about 98%, about 99%, about 100% sequence identity to SEQ ID NO: 7 and/or comprising, consisting essentially of or consisting of SEQ ID NO: 7. In some embodiments, the RNA vectors comprise untranslated regions (UTRs), such as a 5′UTR and/or a 3′ UTR. In some embodiments, the RNA vectors include a 5′ UTR that has at least about 80%, about 85%, about 90%, about 95%, about 96%, about 97%, about 98%, about 99%, about 100% sequence identity to SEQ ID NO: 8 and/or comprising, consisting essentially of or consisting of SEQ ID NO: 8. In some embodiments, the RNA vectors include a 3′ UTR that has at least about 80%, about 85%, about 90%, about 95%, about 96%, about 97%, about 98%, about 99%, about 100% sequence identity to SEQ ID NO: 9 and/or comprising, consisting essentially of or consisting of SEQ ID NO: 9. In some embodiments, the RNA vectors may also include linker regions, and/or cap regions. In some embodiments, the RNA vectors provided herein comprise at least one modified base pair, such as an N1-methyl-pseudo-uridine-triphosphate.

In another aspect, a method of treating a cell, tissue or organ in a subject in need thereof, comprising contacting the cell, tissue or organ with an RNA vector as described herein is provided. In some embodiments, contacting the cell, tissue or organ with the RNA vector achieves expression of one or more reprogramming factors in the cell, tissue or organ to obtain a rejuvenated cell tissue or organ with retention of cellular identity is provided.

In another aspect, a method for treating a differentiated cell, comprising introducing an RNA vector as described herein is provided. In some embodiments, introducing the RNA vector into the differentiated cell results in expression of one or more reprogramming factors, thereby generating a cell that retains its cellular differentiation and that expresses the one or more reprogramming factor to obtain a rejuvenated cell.

In another aspect, a method of treating an age-related disease or condition, comprising exposing differentiated cells associated with the age-related disease or condition to an RNA vector as described herein is provided. In some embodiments, exposing differentiated cells associated with an age-related disorder with the RNA vector achieves expression of the one or more reprogramming factors in the differentiated cells to obtain rejuvenated cells with retention of cellular identity.

In addition to the exemplary aspects and embodiments described above, further aspects and embodiments will become apparent by reference to the drawings and by study of the following descriptions.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE SEQUENCES

In some embodiments, the methods and compositions for cellular rejuvenation, tissue engineering, and regenerative medicine by transient exposure of cells or tissues to synthetic, non-integrative mRNAs encoding reprogramming factors, comprise exposing the cell, including immune cells, to messenger RNA (mRNA) encoding one or more reprogramming factors wherein the reprogramming factor encoding mRNA encodes a polypeptide encoded by a polynucleotide having at least 95% sequence identity to any one of SEQ ID NOs: 1-19 (Table 1).

TABLE 1

| NAME | SEQUENCE | SEQ ID NO |
|---|---|---|
| OCT4 | ATGGCTGGCCATCTCGCAAGTGACTTCG | 1 |
| | CATTTTCCCCGCCCCCAGGCGGCGGTGG | |
| | AGATGGACCTGGCGGCCCAGAACCAGG | |
| | ATGGGTGGACCCCCGCACGTGGCTTTCT | |
| | TTTCAGGGTCCACCAGGCGGACCCGGTA | |
| | TTGGACCCGGCGTGGGTCCTGGGTCAGA | |
| | AGTCTGGGGTATCCCACCCTGTCCCCCA | |
| | CCATACGAATTTTGCGGCGGAATGGCCT | |
| | ATTGCGGCCCTCAAGTCGGGGTCGGTCT | |
| | GGTACCTCAGGGTGGTCTGGAAACATCC | |
| | CAACCAGAAGGTGAGGCCGGTGTGGGA | |
| | GTTGAATCCAATTCTGACGGAGCATCTC | |
| | CAGAACCTTGTACTGTGACACCAGGAGC | |
| | TGTTAAATTGGAGAAAGAAAAGCTCGA | |
| | ACAGAATCCAGAAGAATCACAAGATATT | |
| | AAGGCGCTCCAAAAGGAGCTGGAACAG | |
| | TTCGCGAAACTTCTTAAACAGAAACGCA | |
| | TTACTCTCGGGTACACCCAAGCGGACGT | |
| | TGGACTGACTCTCGGTGTGCTGTTCGGC | |
| | AAAGTCTTTAGTCAGACCACAATATGTC | |
| | GATTCGAAGCCCTTCAACTGTCATTTAA | |
| | GAATATGTGCAAACTTCGACCTCTGCTC | |
| | CAGAAATGGGTCGAAGAGGCGGATAAT | |
| | AACGAGAACCTGCAAGAAATCTGTAAG | |
| | GCGGAGACTCTGGTTCAAGCTCGCAAAA | |
| | GGAAACGTACGTCTATAGAAAATAGAGT | |
| | CCGTGGGAATCTTGAAAACCTGTTTCTC | |
| | CAATGTCCAAAGCCTACTTTGCAACAAA | |
| | TATCTCATATTGCGCAACAACTCGGCCT | |
| | GGAAAAGGACGTAGTTAGAGTCTGGTTT | |
| | TGCAATCGCAGACAGAAAGGGAAACGG | |
| | TCTTCCAGTGATTACGCGCAGAGGGAAG | |
| | ACTTCGAAGCAGCCGGTTCACCGTTTTC | |
| | CGGCGGCCCGGTATCTTTCCCATTGGCT | |
| | CCCGGTCCTCACTTCGGCACACCCGGGT | |
| | ACGGCTCACCACATTTTACCGCCCTTTAT | |
| | TCAAGCGTTCCCTTTCCGGAAGGCGAGG | |
| | CTTTCCCGCCGGTGTCAGTGACTACACTT | |
| | GGATCCCCAATGCACAGCAATTAG | |
| SOX2 | ATGTATAATATGATGGAAACCGAATTGA | 2 |
| | AACCACCCGGGCCCCAACAGACCTCTGG | |
| | CGGCGGTGGTGGTAATAGCACAGCAGC | |
| | AGCCGCTGGTGGAAATCAAAAGAATTCT | |
| | CCAGATAGAGTGAAACGACCTATGAAC | |
| | GCATTTATGGTCTGGTCTAGAGGACAAC | |
| | GAAGGAAAATGGCTCAAGAAAATCCCA | |
| | AAATGCATAATAGCGAAATTTCCAAACG | |
| | GTTGGGTGCGGAATGGAAGCTCCTCAGC | |
| | GAAACCGAAAAGAGGCCATTTATTGATG | |
| | AAGCGAAAAGACTCAGGGCATTGCATAT | |
| | GAAAGAACATCCAGACTACAAGTATAG | |
| | ACCACGCCGCAAGACAAAGACTCTGATG | |
| | AAGAAGGACAAATATACCCTGCCTGGTG | |
| | GATTGTTGGCTCCTGGCGGTAACAGTAT | |
| | GGCTTCTGGCGTGGGCGTTGGGGCTGGA | |
| | CTTGGTGCCGGGGTCAATCAACGAATGG | |
| | ATTCCTATGCCCATATGAATGGATGGAG | |
| | TAATGGTTCCTATTCTATGATGCAAGAT | |
| | CAATTGGGATATCCTCAACATCCCGGTC | |
| | TGAACGCTCATGGTGCTGCTCAAATGCA | |
| | ACCTATGCATCGGTATGATGTAAGTGCA | |
| | TTGCAATATAATAGCATGACATCCAGTC | |
| | AAACATATATGAATGGGTCACCAACATA | |
| | TAGTATGAGCTATTCCCAACAAGGTACA | |
| | CCAGGGATGGCCCTGGGGAGCATGGGG | |
| | AGTGTCGTTAAAAGTGAAGCTTCAAGTT | |
| | CACCACCCGTAGTGACGAGTTCTTCACA | |
| | TTCTCGAGCCCCATGTCAAGCAGGAGAT | |
| | CTTAGGGATATGATTTCAATGTACTTGC | |
| | CAGGGGCTGAAGTCCCCGAGCCGGCAG | |
| | CGCCTTCTAGGCTGCATATGTCTCAACA | |
| | TTATCAATCCGGACCCGTTCCAGGGACC | |
| | GCTATCAATGGTACGTTGCCATTGTCCC | |
| | ATATGTAA | |
| C-Myc | ATGGACTTCTTTCGAGTGGTAGAGAATC | 3 |
| | AACAACCCCCGGCCACCATGCCATTGAA | |
| | TGTATCATTTACAAACCGGAACTACGAC | |
| | CTGGACTATGATTCAGTTCAGCCTTACTT | |
| | TTATTGTGACGAAGAAGAAAATTTCTAT | |
| | CAACAACAACAACAATCAGAATTGCAA | |
| | CCACCCGCTCCATCAGAAGACATTTGGA | |
| | AGAAGTTTGAACTCCTGCCAACTCCACC | |
| | GCTCAGCCCGAGTAGACGTTCTGGACTG | |
| | TGTTCTCCTTCTTATGTGGCTGTGACTCC | |
| | GTTTTCACTGCGTGGCGATAATGATGGC | |
| | GGCGGTGGCTCTTTTAGTACAGCAGATC | |
| | AACTTGAAATGGTCACAGAACTCCTTGG | |
| | TGGTGATATGGTTAATCAATCATTCATTT | |
| | GTGATCCCGATGATGAGACATTTATAAA | |
| | GAACATCATCATACAAGACTGCATGTGG | |
| | TCAGGGTTTAGTGCTGCTGCGAAACTGG | |
| | TGAGCGAAAAGTTGGCTTCTTATCAAGC | |
| | CGCCCGGAAGGATAGTGGATCACCAAAT | |
| | CCAGCAAGGGGTCATTCAGTGTGTAGCA | |
| | CAAGCTCTCTGTATCTTCAAGACCTCTCC | |
| | GCGGCTGCAAGTGAATGTATTGATCCAA | |
| | GTGTCGTTTTCCCTTATCCCCTGAATGAT | |
| | TCTTCCTCTCCTAAAAGCTGTGCGAGCC | |
| | AGGATTCTTCAGCTTTCTCCCCAAGCTCC | |
| | GACAGTTTGTTGAGCTCTACTGAAAGTA | |
| | GTCCTCAAGGGTCACCGGAACCTCTCGT | |
| | CCTTCACGAAGAAACACCCCCTACAACT | |
| | AGTTCCGATTCCGAAGAAGAACAGGAA | |
| | GACGAAGAGGAGATTGACGTGGTATCA | |
| | GTTGAGAAAAGACAAGCCCCCGGGAAG | |
| | CGAAGCGAAAGCGGGAGCCCAAGCGCC | |
| | GGCGGACATTCCAAGCCCCCACATTCTC | |
| | CTTTGGTACTGAAAAGATGTCATGTGAG | |
| | CACCCACCAACATAATTATGCTGCTCCC | |
| | CCATCAACCAGGAAAGATTACCCCGCCG | |
| | CTAAACGAGTTAAACTGGATTCAGTGAG | |
| | GGTTCTTAGGCAAATTTCAAATAATAGG | |
| | AAGTGTACTTCACCTCGCAGTAGCGATA | |
| | CAGAAGAAAACGTTAAAAGACGGACGC | |
| | ATAATGTGCTGGAACGACAAAGACGAA | |
| | ATGAACTTAAGAGGTCCTTCTTCGCGCT | |
| | TAGGGATCAAATACCTGAACTGGAGAAT | |
| | AACGAGAAAGCTCCAAAAGTGGTGATTT | |
| | TGAAGAAGGCTACTGCGTATATACTTTC | |
| | TGTACAGGCCGAAGAACAGAAACTGAT | |
| | ATCAGAGGAAGATCTGCTTCGTAAGCGC | |
| | AGAGAGCAACTGAAGCATAAGCTCGAG | |
| | CAACTCCGCAATAGCTGCGCCTAG | |
| KLF4 | ATGCGGCAGCCACCAGGGGAAAGTGAT | 4 |
| | ATGGCCGTTTCCGACGCTCTTCTGCCTTC | |
| | ATTTTCAACCTTTGCTTCCGGACCTGCCG | |
| | GGCGGGAAAAGACGCTTAGGCAGGCCG | |
| | GAGCACCCAACAATCGATGGAGAGAAG | |
| | AACTGAGCCATATGAAAAGACTGCCGCC | |
| | TGTACTCCCGGGGCGGCCATACGATCTC | |
| | GCCGCCGCTACAGTAGCAACTGATTTGG | |
| | AATCCGGTGGTGCTGGGGCAGCCTGTGG | |
| | CGGATCTAATCTTGCTCCTCTGCCAAGA | |
| | CGGGAAACGGAAGAATTTAATGACCTGC | |
| | TCGATTTGGATTTCATCCTTTCTAACTCA | |
| | CTTACACACCCACCAGAAAGTGTCGCTG | |
| | CTACGGTCTCTAGCTCTGCCAGCGCAAG | |
| | CAGTTCATCTTCTCCATCTTCCTCAGGAC | |
| | CCGCAAGTGCCCCGAGCACTTGTTCCTT | |
| | TACGTACCCAATACGAGCTGGCAATGAT | |
| | CCTGGAGTTGCTCCCGGTGGTACAGGCG | |
| | GTGGTCTGTTGTACGGAAGAGAATCAGC | |
| | CCCACCACCAACCGCACCATTTAATTTG | |
| | GCCGATATTAATGATGTTTCACCTAGTG | |
| | GTGGTTTTGTTGCGGAACTGCTCCGTCCC | |
| | GAGCTGGATCCCGTCTATATCCCACCAC | |
| | AACAACCCCAACCACCTGGCGGCGGATT | |
| | GATGGGTAAATTTGTTCTTAAAGCATCC | |
| | CTTTCCGCACCAGGGTCAGAATATGGAA | |
| | GTCCCTCAGTGATTTCCGTATCTAAGGG | |
| | ATCCCCCGATGGGTCCCATCCAGTTGTC | |
| | GTTGCCCCTTATAATGGTGGCCCGCCTA | |
| | GAACCTGTCCGAAAATAAAACAAGAAG | |
| | CTGTATCATCATGTACGCATCTCGGGGC | |
| | AGGTCCACCCCTGTCTAACGGGCATCGC | |
| | CCTGCAGCCCATGATTTTCCTTTGGGCCG | |
| | ACAACTGCCGTCCCGTACAACTCCAACA | |
| | CTCGGCCTCGAAGAGGTCCTCAGTAGTA | |
| | GAGATTGCCATCCAGCACTCCCCCTGCC | |
| | ACCAGGTTTTCACCCTCATCCCGGTCCTA | |
| | ACTATCCCAGCTTTCTCCCAGACCAAAT | |
| | GCAACCCCAGGTTCCCCCACTTCACTAT | |
| | CAGGGCCAAAGCAGAGGTTTCGTGGCGC | |
| | GAGCCGGAGAACCATGCGTCTGTTGGCC | |
| | TCATTTTGGCACCCATGGAATGATGTTG | |
| | ACACCCCCAAGTAGTCCACTCGAACTGA | |
| | TGCCCCCTGGCAGTTGTATGCCTGAAGA | |
| | ACCTAAACCCAAAAGAGGGCGGCGGAG | |
| | TTGGCCACGAAAGCGAACAGCAACTCAT | |
| | ACCTGCGACTATGCTGGATGTGGGAAGA | |
| | CATATACCAAATCTAGCCACCTGAAAGC | |
| | GCATCTCCGCACGCATACTGGAGAAAAG | |
| | CCCTATCATTGCGATTGGGATGGGTGCG | |
| | GCTGGAAGTTTGCACGATCTGACGAGCT | |
| | TACTAGACATTATCGCAAGCATACCGGA | |
| | CATCGGCCCTTTCAATGTCAGAAGTGTG | |
| | ATCGCGCCTTCAGCCGGAGCGATCATCT | |
| | GGCTCTGCATATGAAACGACACTTCTAG | |
| LIN28 | ATGGGCTCAGTCAGCAATCAACAATTCG | 5 |
| | CGGGTGGATGTGCAAAAGCTGCTGAGG | |
| | AAGCCCCAGAAGAAGCTCCAGAAGATG | |
| | CCGCTCGCGCCGCTGATGAACCACAACT | |
| | GCTCCATGGAGCTGGGATTTGCAAATGG | |
| | TTTAATGTCCGGATGGGCTTTGGTTTCTT | |
| | GTCTATGACAGCAAGAGCTGGAGTGGCC | |
| | CTCGATCCACCCGTCGACGTGTTCGTAC | |
| | ATCAATCTAAACTTCATATGGAAGGATT | |
| | TCGCTCACTCAAAGAAGGAGAAGCCGTA | |
| | GAATTTACATTCAAGAAAAGTGCGAAAG | |
| | GGCTTGAGAGCATACGGGTGACAGGGC | |
| | CAGGCGGCGTGTTTTGCATCGGATCAGA | |
| | ACGAAGGCCCAAGGGTAAATCCATGCA | |
| | AAAGAGGCGATCCAAAGGGGATCGGTG | |
| | TTATAATTGCGGTGGCCTTGACCACCAC | |
| | GCGAAAGAGTGTAAACTTCCCCCGCAAC | |
| | CTAAGAAATGTCATTTCTGTCAATCCAT | |
| | ATCACACATGGTGGCGAGCTGCCCATTG | |
| | AAAGCACAACAAGGACCGTCCGCCCAA | |
| | GGGAAACCTACTTATTTCAGGGAAGAGG | |
| | AAGAGGAGATTCATAGTCCCACTCTCCT | |
| | TCCTGAAGCCCAAAACTAG | |
| NANOG | ATGTCAGTCGACCCGGCCTGCCCACAGA | 6 |
| | GTCTGCCCTGTTTCGAGGCTTCAGATTGC | |
| | AAGGAGTCCTCTCCGATGCCCGTCATCT | |
| | GCGGACCCGAGGAGAATTACCCCAGTCT | |
| | GCAGATGTCCAGTGCCGAAATGCCACAT | |
| | ACAGAAACGGTTTCACCGCTCCCATCTT | |
| | CAATGGACCTTTTGATCCAAGATTCTCC | |
| | CGACAGCAGCACTTCCCCAAAGGGAAA | |
| | GCAGCCTACCTCAGCGGAGAAATCTGTG | |
| | GCCAAGAAAGAGGATAAAGTTCCTGTTA | |
| | AGAAGCAAAAGACAAGGACCGTATTTTC | |
| | CTCAACACAACTCTGCGTCCTTAACGAC | |
| | CGGTTCCAACGCCAAAAGTATCTGTCCT | |
| | TGCAACAAATGCAGGAGCTGAGCAATAT | |
| | TTTGAATCTGTCTTATAAGCAAGTCAAG | |
| | ACTTGGTTTCAAAACCAGCGTATGAAGA | |
| | GTAAAAGATGGCAGAAGAATAATTGGC | |
| | CCAAGAACTCTAACGGCGTTACTCAAAA | |
| | GGCGAGTGCCCCCACTTATCCATCTCTG | |
| | TATTCATCATATCATCAAGGCTGTTTGGT | |
| | TAATCCCACGGGCAATCTGCCGATGTGG | |
| | TCCAATCAAACGTGGAATAACAGTACTT | |
| | GGTCCAATCAAACTCAAAATATACAAAG | |
| | TTGGTCAAATCATTCTTGGAATACCCAA | |
| | ACGTGGTGTACTCAGAGCTGGAATAACC | |
| | AAGCATGGAACTCCCCATTTTACAATTG | |
| | CGGCGAAGAGTCACTCCAAAGCTGTATG | |
| | CAATTTCAACCCAACAGTCCCGCATCCG | |
| | ATCTTGAAGCAGCACTGGAAGCGGCCGG | |
| | TGAGGGGCTGAACGTGATTCAACAAACA | |
| | ACAAGATACTTCTCCACACCTCAGACTA | |
| | TGGACCTTTTCCTGAATTATTCAATGAAT | |
| | ATGCAGCCCGAGGATGTCTAG | |
| Exemplary Tail | AAAAAAAAAAAAAAAAAAAAAAAAAA | 7 |
| | AAAAAAAAAAAAAAAAAAAAAAAAAA | |
| | GCATAGCAGCTGACTAAAAAAAAAAAA | |
| | AAAAAAAAAAAAAAAAAAAAAAAAAA | |
| | AAAAAAAAAA | |
| Exemplary 5′ UTR | TTGGACCCTCGTACAGAAGCTAATACGA | 8 |
| | CTCACTATAGGGAAATAAGAGAGAAAA | |
| | GAAGAGTAAGAAGAAATATAAGAGCCA | |
| | CCATG | |
| Exemplary 3′ UTR | GCTGCCTTCTGCGGGGCTTGCCTTCTGGC | 9 |
| | CATGCCCTTCTTCTCTCCCTTGCACCTGT | |
| | ACCTCTTGGTCTTTGAATAAAGCCTGAG | |
| | TAGGAAGTGAGGGTCTAGAACTAGTGTC | |
| | GACGC | |
| GLIS1 | ATGGCCGAAGCCAGAACATCTCTGTCTG | 10 |
| | CCCACTGTAGAGGCCCCCTGGCTACAGG | |
| | TCTGCACCCCGACCTGGATCTGCCTGGC | |
| | CGGAGCTTGGCCACCCCTGCTCCTAGCT | |
| | GCTACCTGCTGGGCTCTGAACCCAGCTC | |
| | CGGCCTTGGCCTGCAGCCTGAGACACAC | |
| | CTGCCTGAAGGCTCCCTCAAGCGGTGCT | |
| | GCGTGCTGGGCCTGCCTCCAACATCCCC | |
| | TGCTTCCTCTTCTCCATGCGCCAGCAGCG | |
| | ACGTGACCAGCATCATCAGAAGCAGCCA | |
| | GACAAGCCTCGTGACCTGCGTGAACGGC | |
| | CTGCGGAGCCCTCCCCTGACCGGCGATC | |
| | TGGGAGGCCCCTCAAAGAGAGCCCGGC | |
| | CCGGCCCTGCCAGCACCGACAGCCACGA | |
| | GGGCAGCCTGCAGCTGGAAGCTTGTAGA | |
| | AAGGCCAGCTTCCTGAAACAGGAGCCTG | |
| | CCGACGAGTTCTCCGAGCTGTTCGGCCC | |
| | CCACCAGCAGGGACTGCCGCCTCCTTAT | |
| | CCCCTGTCCCAGCTGCCCCCAGGACCAA | |
| | GTCTGGGAGGACTCGGCCTGGGACTGGC | |
| | CGGCAGAGTAGTGGCCGGAAGACAGGC | |
| | CTGCCGGTGGGTGGACTGTTGTGCCGCT | |
| | TACGAGCAGCAGGAGGAACTGGTGCGC | |
| | CACATCGAGAAAAGCCACATCGACCAG | |
| | AGGAAGGGCGAGGATTTCACATGCTTCT | |
| | GGGCCGGCTGCGTGCGGCGGTACAAGCC | |
| | CTTTAATGCCAGATACAAGCTGCTGATC | |
| | CACATGAGAGTGCACAGCGGCGAAAAG | |
| | CCTAACAAGTGCATGTTCGAGGGCTGCA | |
| | GCAAGGCCTTCTCTAGACTGGAAAATCT | |
| | GAAAATCCACCTGAGATCTCACACCGGA | |
| | GAAAAGCCTTACCTGTGCCAGCATCCTG | |
| | GATGTCAGAAGGCCTTCAGCAACAGCTC | |
| | TGATAGAGCCAAGCACCAGAGAACCCA | |
| | CCTGGACACCAAGCCTTATGCCTGCCAG | |
| | ATCCCTGGCTGCAGCAAAAGATACACCG | |
| | ATCCTTCTAGCCTGCGGAAGCACGTGAA | |
| | GGCCCATAGCGCTAAGGAACAGCAGGT | |
| | GCGCAAAAAGCTGCATGCCGGCCCAGAT | |
| | ACCGAGGCCGATGTGCTAACTGAGTGCC | |
| | TGGTCCTGCAACAGCTGCACACATCTAC | |
| | CCAGCTGGCCGCCAGCGACGGCAAGGG | |
| | CGGATGTGGCCTGGGACAGGAGCTGCTG | |
| | CCTGGTGTTTACCCCGGAAGCATCACCC | |
| | CTCACAACGGCCTGGCCAGCGGACTGCT | |
| | GCCCCCCGCACACGACGTGCCTAGCCGG | |
| | CATCACCCTCTGGACGCCACCACCAGCA | |
| | GCCACCACCACCTGAGCCCCCTGCCTAT | |
| | GGCCGAAAGCACCAGAGATGGCCTCGG | |
| | CCCTGGCCTCCTCAGCCCTATCGTGTCTC | |
| | CACTGAAAGGCCTGGGCCCCCCTCCTCT | |
| | TCCTCCAAGCAGCCAGAGCCACAGCCCC | |
| | GGCGGCCAGCCTTTCCCTACCCTGCCTTC | |
| | TAAGCCCAGCTACCCTCCTTTTCAGAGC | |
| | CCTCCTCCCCCTCCACTGCCCTCTCCTCA | |
| | GGGCTACCAAGGCAGCTTCCACTCTATC | |
| | CAGTCCTGTTTTCCTTACGGCGACTGCTA | |
| | CAGAATGGCCGAACCAGCCGCCGGCGG | |
| | CGACGGCCTGGTGGGCGAGACACACGG | |
| | CTTTAACCCCCTGAGACCTAACGGCTAC | |
| | CACTCTCTGAGCACACCTCTGCCTGCTA | |
| | CCGGCTACGAGGCTCTGGCAGAGGCCAG | |
| | CTGCCCTACCGCCCTGCCGCAACAACCT | |
| | AGCGAAGACGTCGTGTCCTCTGGCCCAG | |
| | AGGACTGCGGATTCTTCCCCAACGGCGC | |
| | CTTCGACCACTGCCTGGGCCACATTCCT | |
| | AGCATCTACACCGACACATGA | |
| OCT4MyoD for T- | ATGGAGTTCGCCATGGAGCTGCTGTCGC | 11 |
| cells (T- | CACCGTTGCGTGACGTGGACCTGACAGC | |
| OCT4MyoD) | CCCCGACGGCTCTCTGTGCTCCTTTGCTA | |
| | CTACCGACGATTTCTACGATGACCCGTG | |
| | CTTTGATTCTCCCGACCTGCGCTTTTTCG | |
| | AAGACTTAGATCCGCGCCTGATGCATGT | |
| | AGGTGCTCTGCTAAAGCCCGAGGAGCAC | |
| | ATGGCTGGCCACTTGGCTTCCGACTTCG | |
| | CGTTCTCCCCGCCACCCGGCGGCGGAGG | |
| | CGATGGCCCTGGCGGACCCGAGCCAGGT | |
| | TGGGTCGACCCTCGGACCTGGCTCTCCT | |
| | TCCAGGGCCCCCCAGGCGGGCCCGGCAT | |
| | AGGTCCCGGCGTTGGACCCGGGAGCGA | |
| | GGTGTGGGGCATCCCTCCGTGCCCACCC | |
| | CCGTATGAGTTCTGCGGGGGTATGGCCT | |
| | ACTGCGGGCCTCAGGTGGGCGTCGGCCT | |
| | CGTCCCTCAGGGGGGTCTGGAGACCTCT | |
| | CAGCCGGAGGGAGAGGCTGGGGTCGGC | |
| | GTGGAGAGCAACTCCGACGGGGCGTCG | |
| | CCCGAACCTTGCACTGTCACGCCCGGGG | |
| | CCGTTAAGCTGGAGAAGGAGAAACTTG | |
| | AGCAGAACCCCGAGGAGAGCCAGGACA | |
| | TCAAGGCGCTGCAGAAGGAGCTGGAAC | |
| | AGTTCGCCAAGCTGCTGAAGCAGAAGCG | |
| | CATCACCCTAGGTTACACCCAGGCGGAC | |
| | GTGGGCCTGACGCTTGGTGTGCTGTTCG | |
| | GAAAGGTGTTCAGCCAGACGACCATCTG | |
| | CCGATTCGAGGCCCTCCAGCTGTCCTTC | |
| | AAGAACATGTGCAAGTTGCGGCCCCTGC | |
| | TCCAAAAATGGGTGGAGGAGGCTGACA | |
| | ACAACGAGAATCTCCAGGAGATCTGTAA | |
| | AGCCGAGACTCTGGTGCAGGCCCGCAAA | |
| | CGCAAGCGTACCTCGATTGAAAACAGGG | |
| | TGCGTGGCAACCTGGAGAACCTGTTCCT | |
| | ACAGTGTCCCAAGCCTACCCTTCAGCAG | |
| | ATTAGCCACATCGCACAACAGTTGGGCC | |
| | TTGAAAAGGATGTGGTACGCGTGTGGTT | |
| | CTGTAACCGCCGCCAGAAGGGTAAGCGC | |
| | AGCTCCAGCGACTACGCGCAGAGAGAG | |
| | GACTTTGAGGCTGCAGGATCTCCTTTTTC | |
| | TGGCGGCCCTGTGAGTTTCCCTCTGGCC | |
| | CCGGGACCCCACTTTGGTACTCCGGGCT | |
| | ACGGCTCCCCGCACTTCACCGCCCTGTA | |
| | CTCTAGTGTCCCGTTTCCCGAGGGCGAG | |
| | GCGTTCCCCCCAGTGTCCGTGACCACAC | |
| | TGGGGTCCCCAATGCATTCAAATTGA | |
| B18R for T cells | ATGACAATGAAAATGATGGTGCACATCT | 12 |
| (T-B18R) | ACTTCGTCTCTCTTCTGCTGCTGCTGTTT | |
| | CACAGCTACGCAATTGACATCGAAAACG | |
| | AGATCACCGAGTTCTTCAACAAGATGCG | |
| | TGACACCCTTCCCGCCAAGGATTCTAAA | |
| | TGGCTCAACCCGGCCTGCATGTTTGGAG | |
| | GCACCATGAACGACATCGCGGCGCTGGG | |
| | CGAGCCCTTCTCCGCTAAATGTCCCCCG | |
| | ATCGAAGATTCTCTGCTGAGCCACCGCT | |
| | ACAAGGACTATGTGGTCAAATGGGAGC | |
| | GCCTGGAGAAGAACCGTCGGCGCCAGG | |
| | TCTCAAACAAGCGCGTAAAGCATGGAG | |
| | ATCTTTGGATCGCCAACTACACCAGTAA | |
| | ATTTAGCAACCGCCGCTACCTGTGCACT | |
| | GTCACCACCAAGAATGGGGACTGCGTGC | |
| | AGGGCATCGTGCGGAGCCACATCCGCAA | |
| | GCCTCCCTCTTGTATTCCCAAGACCTACG | |
| | AGCTGGGGACACATGACAAGTACGGCA | |
| | TTGACCTGTATTGCGGGATCCTGTACGC | |
| | GAAGCACTACAACAACATCACCTGGTAC | |
| | AAGGACAACAAGGAGATTAACATCGAT | |
| | GACATCAAGTACTCCCAGACTGGCAAGG | |
| | AGCTCATCATCCACAACCCTGAGCTGGA | |
| | GGACTCCGGTAGATATGATTGTTACGTG | |
| | CATTACGACGATGTGCGCATCAAAAACG | |
| | ACATTGTCGTTTCCAGGTGTAAGATACT | |
| | CACTGTGATCCCTAGTCAGGACCACCGC | |
| | TTCAAGCTGATCTTGGACCCGAAGATAA | |
| | ATGTGACCATCGGGGAGCCAGCCAATAT | |
| | CACCTGCACTGCCGTGTCCACCTCGTTG | |
| | CTCATTGACGATGTACTGATTGAGTGGG | |
| | AGAATCCATCGGGTTGGCTAATCGGCTT | |
| | CGACTTCGACGTGTACTCGGTGCTAACC | |
| | TCCCGTGGTGGCATCACTGAGGCTACGC | |
| | TTTATTTCGAGAACGTGACGGAGGAGTA | |
| | CATCGGCAACACCTACAAGTGCCGAGGC | |
| | CACAACTACTACTTCGAAAAAACCCTGA | |
| | CGACGACCGTGGTTTTGGAGTGA | |
| KLF4 for T cells | ATGCGTCAGCCACCCGGGGAGAGCGAC | 13 |
| (T-KLF4) | ATGGCCGTGTCGGACGCGCTGCTGCCAT | |
| | CCTTTTCCACCTTCGCCTCGGGTCCGGCC | |
| | GGCCGAGAGAAGACTCTGCGCCAGGCC | |
| | GGAGCCCCTAACAACCGCTGGAGAGAG | |
| | GAGCTGTCACACATGAAACGCCTGCCCC | |
| | CCGTGCTGCCTGGGCGCCCCTACGACCT | |
| | TGCCGCGGCCACGGTGGCTACCGACTTG | |
| | GAGTCTGGAGGTGCTGGAGCAGCGTGTG | |
| | GCGGAAGCAACCTGGCACCGTTGCCACG | |
| | CCGGGAGACCGAGGAGTTCAACGACTTG | |
| | TTGGATCTGGACTTTATTCTGTCCAACTC | |
| | CCTTACACACCCGCCCGAGAGCGTAGCA | |
| | GCCACCGTGAGCTCCAGTGCTTCCGCTT | |
| | CCTCCTCATCCAGCCCGTCGTCTTCTGGC | |
| | CCTGCCTCTGCGCCGTCGACCTGTTCGTT | |
| | CACCTATCCCATCCGGGCCGGCAACGAT | |
| | CCGGGCGTGGCCCCGGGCGGCACCGGTG | |
| | GTGGTCTCCTGTACGGCAGGGAGTCCGC | |
| | CCCCCCTCCAACCGCTCCCTTCAACCTCG | |
| | CGGACATCAATGACGTGTCCCCCTCTGG | |
| | CGGCTTCGTTGCAGAACTGTTAAGGCCT | |
| | GAACTGGATCCAGTGTACATCCCGCCCC | |
| | AGCAGCCTCAGCCGCCGGGCGGCGGTCT | |
| | GATGGGCAAATTTGTCCTGAAGGCGTCT | |
| | CTGTCTGCTCCTGGCTCCGAGTACGGCA | |
| | GCCCCAGTGTGATTAGCGTGTCTAAAGG | |
| | CAGCCCCGACGGGTCGCACCCCGTGGTG | |
| | GTCGCTCCTTACAACGGTGGACCCCCGC | |
| | GCACCTGCCCAAAGATCAAGCAGGAGG | |
| | CTGTTTCTTCATGCACTCATCTAGGCGCC | |
| | GGTCCACCCCTTTCCAATGGCCACCGGC | |
| | CCGCCGCGCATGACTTCCCCCTGGGCCG | |
| | CCAGCTGCCCAGCCGGACCACACCTACC | |
| | TTAGGCCTGGAGGAGGTGCTTAGTTCGC | |
| | GCGACTGTCATCCTGCCCTGCCTCTCCCT | |
| | CCAGGCTTCCACCCCCACCCGGGACCCA | |
| | ACTACCCGTCCTTCCTGCCCGACCAGAT | |
| | GCAACCTCAGGTCCCTCCCCTGCACTAC | |
| | CAGGGACAGAGCCGCGGTTTCGTCGCCC | |
| | GTGCTGGCGAGCCATGCGTCTGTTGGCC | |
| | GCACTTCGGCACGCATGGAATGATGCTA | |
| | ACTCCCCCGAGCTCCCCCCTGGAGCTGA | |
| | TGCCCCCGGGTTCCTGTATGCCAGAGGA | |
| | GCCCAAGCCTAAGCGCGGCAGACGTAGT | |
| | TGGCCCCGTAAGCGCACCGCCACACATA | |
| | CGTGCGACTACGCGGGGTGCGGGAAAA | |
| | CCTACACCAAGTCTTCTCACCTCAAGGC | |
| | TCACTTGCGTACTCACACTGGAGAGAAG | |
| | CCTTATCACTGCGACTGGGATGGGTGCG | |
| | GGTGGAAGTTTGCTCGCTCCGACGAACT | |
| | CACCCGCCATTACCGCAAGCACACCGGC | |
| | CACCGCCCCTTCCAGTGTCAGAAGTGCG | |
| | ATCGAGCGTTCTCCCGCTCGGACCACCT | |
| | GGCCCTACACATGAAGAGGCACTTTTGA | |
| LIN28 for T cells | ATGGGCTCCGTGTCGAATCAGCAGTTCG | 14 |
| (T-LIN28) | CAGGCGGGTGCGCCAAGGCAGCCGAGG | |
| | AGGCCCCGGAGGAGGCTCCTGAAGACG | |
| | CCGCTCGCGCGGCGGACGAGCCTCAGCT | |
| | ACTCCATGGAGCTGGCATCTGCAAATGG | |
| | TTCAACGTGCGAATGGGATTTGGCTTCC | |
| | TGTCTATGACTGCTCGTGCCGGCGTGGC | |
| | GCTGGACCCGCCCGTAGACGTGTTCGTC | |
| | CACCAGAGCAAGCTGCATATGGAGGGTT | |
| | TCAGATCTCTGAAGGAGGGCGAGGCCGT | |
| | GGAGTTCACGTTCAAGAAGTCGGCCAAA | |
| | GGTCTGGAGAGCATCCGCGTCACCGGTC | |
| | CCGGCGGCGTTTTTTGTATTGGCTCCGA | |
| | GCGCCGGCCCAAGGGCAAGTCCATGCA | |
| | GAAGCGCCGCTCCAAGGGGGACAGGTG | |
| | TTACAACTGCGGGGGTTTGGATCACCAC | |
| | GCGAAGGAGTGCAAACTTCCTCCTCAGC | |
| | CGAAGAAATGTCACTTTTGTCAGAGCAT | |
| | CAGTCACATGGTGGCTTCATGCCCACTC | |
| | AAGGCCCAACAGGGACCCTCTGCGCAG | |
| | GGCAAGCCCACCTACTTCCGCGAGGAGG | |
| | AAGAAGAGATCCACTCCCCAACCCTGCT | |
| | GCCCGAGGCTCAGAACTGA | |
| NANOG for T cells | ATGTCTGTTGACCCGGCCTGCCCACAGA | 15 |
| (T-NANOG) | GCCTTCCCTGCTTTGAGGCTTCCGACTGT | |
| | AAAGAGAGTTCCCCGATGCCCGTGATTT | |
| | GTGGTCCTGAAGAGAACTACCCGTCCCT | |
| | ACAGATGTCATCGGCGGAGATGCCTCAT | |
| | ACCGAGACCGTGTCCCCCTTACCCTCTTC | |
| | TATGGATCTGCTGATCCAGGACTCGCCC | |
| | GACAGCTCGACCAGCCCTAAGGGCAAG | |
| | CAGCCAACCTCCGCAGAGAAGTCCGTGG | |
| | CTAAGAAGGAGGACAAAGTACCGGTGA | |
| | AGAAACAGAAGACTCGCACCGTGTTCTC | |
| | GTCGACCCAGCTGTGCGTGCTCAACGAC | |
| | AGGTTTCAGCGCCAGAAGTACCTGTCCC | |
| | TGCAGCAGATGCAAGAGCTCAGCAACAT | |
| | CCTGAACCTGAGCTACAAGCAGGTCAAG | |
| | ACCTGGTTCCAGAACCAGCGTATGAAGT | |
| | CCAAGCGGTGGCAAAAAAACAACTGGC | |
| | CCAAGAACAGCAACGGTGTCACCCAGA | |
| | AGGCCTCCGCCCCCACCTACCCGTCTCTT | |
| | TACTCCAGCTATCACCAGGGCTGCCTGG | |
| | TGAATCCAACAGGAAACCTGCCCATGTG | |
| | GTCTAATCAGACCTGGAACAACTCCACC | |
| | TGGTCTAACCAGACACAGAACATCCAGA | |
| | GTTGGTCTAATCACTCTTGGAACACGCA | |
| | GACCTGGTGCACTCAGAGTTGGAATAAC | |
| | CAGGCCTGGAATTCCCCCTTCTACAACT | |
| | GCGGGGAGGAGAGCTTGCAGTCATGTAT | |
| | GCAGTTCCAGCCCAACTCCCCAGCCAGT | |
| | GACCTGGAGGCCGCGCTGGAGGCGGCT | |
| | GGCGAGGGCCTCAACGTGATCCAACAG | |
| | ACGACCCGCTACTTCTCCACGCCTCAGA | |
| | CTATGGATTTGTTCCTGAACTATTCGATG | |
| | AACATGCAGCCTGAAGATGTGTGA | |
| OCT4 for T cells | ATGGCGGGCCACTTGGCTTCCGACTTCG | 16 |
| (T-OCT4) | CGTTCTCTCCCCCGCCTGGCGGAGGCGG | |
| | GGACGGCCCCGGCGGCCCTGAGCCAGG | |
| | CTGGGTCGATCCACGCACCTGGCTCTCC | |
| | TTCCAGGGACCCCCCGGGGGGCCCGGCA | |
| | TCGGTCCCGGGGTAGGCCCCGGATCGGA | |
| | GGTGTGGGGCATCCCGCCGTGCCCACCG | |
| | CCCTATGAGTTCTGTGGGGGTATGGCCT | |
| | ACTGCGGGCCTCAGGTCGGTGTCGGTCT | |
| | GGTGCCTCAGGGCGGTCTCGAGACCAGC | |
| | CAGCCGGAGGGCGAGGCTGGCGTGGGT | |
| | GTGGAGAGCAACAGCGATGGCGCTTCTC | |
| | CTGAACCATGCACTGTCACCCCCGGCGC | |
| | CGTGAAGCTGGAGAAAGAGAAACTGGA | |
| | GCAGAATCCAGAGGAGAGTCAGGACAT | |
| | CAAGGCCCTGCAGAAGGAACTGGAACA | |
| | GTTCGCCAAGCTGCTGAAGCAGAAGCGT | |
| | ATCACCCTTGGATACACCCAGGCGGACG | |
| | TGGGCCTCACTTTAGGCGTTCTTTTCGGA | |
| | AAGGTGTTCTCACAGACCACAATCTGCC | |
| | GATTCGAGGCCCTGCAACTTTCTTTCAA | |
| | AAACATGTGCAAGTTGCGGCCCCTACTG | |
| | CAGAAATGGGTGGAGGAAGCGGACAAC | |
| | AACGAGAATCTGCAGGAGATCTGTAAG | |
| | GCCGAGACACTGGTGCAGGCTCGCAAGC | |
| | GCAAAAGAACGAGCATTGAGAACCGCG | |
| | TCCGCGGCAACCTGGAGAACCTGTTCCT | |
| | GCAGTGCCCAAAGCCTACCCTCCAGCAA | |
| | ATTAGCCACATCGCTCAGCAGCTGGGCC | |
| | TGGAGAAGGATGTGGTGAGGGTGTGGTT | |
| | CTGTAACCGGCGCCAGAAGGGCAAGCG | |
| | CAGCTCCAGTGACTACGCACAGCGTGAG | |
| | GACTTTGAAGCCGCCGGCTCCCCGTTTT | |
| | CAGGAGGCCCCGTGTCCTTTCCTTTGGCT | |
| | CCCGGCCCTCATTTCGGTACTCCGGGCT | |
| | ACGGCTCCCCCCACTTTACCGCCCTTTAC | |
| | TCCTCTGTCCCCTTCCCCGAGGGAGAGG | |
| | CATTTCCCCCGGTGTCCGTAACGACCCT | |
| | GGGTTCCCCAATGCACTCTAACTGA | |
| SOX2 for T cells | ATGTATAACATGATGGAAACAGAGCTGA | 17 |
| (T-SOX2) | AGCCCCCGGGGCCTCAACAGACCTCCGG | |
| | CGGTGGGGGCGGCAACTCGACCGCTGCC | |
| | GCAGCTGGTGGAAACCAGAAGAACAGT | |
| | CCCGACAGAGTTAAGCGCCCGATGAACG | |
| | CGTTCATGGTGTGGTCTCGCGGCCAGCG | |
| | CCGCAAGATGGCGCAGGAGAATCCAAA | |
| | AATGCACAACTCGGAGATCTCCAAGCGG | |
| | CTCGGTGCCGAGTGGAAGCTGCTAAGCG | |
| | AGACCGAGAAACGTCCTTTTATTGACGA | |
| | GGCCAAGCGCCTGCGTGCGCTTCACATG | |
| | AAGGAGCACCCCGACTACAAGTACAGG | |
| | CCCCGACGCAAAACCAAGACCCTGATGA | |
| | AAAAGGACAAGTACACCCTCCCCGGCG | |
| | GCCTGCTGGCCCCTGGTGGCAACAGCAT | |
| | GGCCTCCGGAGTCGGGGTAGGCGCCGGC | |
| | CTTGGAGCTGGAGTCAACCAGCGTATGG | |
| | ATTCTTACGCGCACATGAATGGGTGGTC | |
| | AAATGGCTCGTATTCTATGATGCAGGAC | |
| | CAGCTGGGCTACCCTCAACACCCCGGCC | |
| | TCAACGCCCATGGAGCGGCTCAGATGCA | |
| | GCCAATGCACCGCTACGATGTGAGCGCC | |
| | CTGCAGTACAACTCTATGACTAGTTCAC | |
| | AGACTTACATGAACGGTTCCCCAACCTA | |
| | CTCCATGTCTTACAGTCAGCAGGGAACG | |
| | CCGGGTATGGCTCTGGGCTCCATGGGCT | |
| | CCGTGGTGAAGTCGGAGGCATCCTCCAG | |
| | CCCTCCCGTGGTCACCTCCTCCTCTCACA | |
| | GCCGCGCTCCTTGCCAGGCCGGGGACCT | |
| | GCGCGACATGATCTCTATGTATCTGCCC | |
| | GGTGCAGAGGTGCCTGAACCGGCGGCCC | |
| | CCTCTCGGTTGCATATGTCCCAGCATTAC | |
| | CAGAGCGGCCCGGTGCCAGGCACTGCCA | |
| | TCAACGGCACCTTGCCCCTGAGCCACAT | |
| | GTGA | |
| cMYC for T-cells | ATGGATTTCTTCCGAGTGGTGGAGAATC | 18 |
| (T-cMyc) | AGCAGCCGCCTGCCACCATGCCCCTTAA | |
| | CGTGTCCTTCACTAACAGAAACTACGAC | |
| | CTGGACTACGACAGTGTCCAGCCCTATT | |
| | TCTACTGTGATGAGGAGGAGAACTTTTA | |
| | CCAGCAGCAACAGCAGAGCGAACTGCA | |
| | GCCCCCAGCGCCCTCGGAGGACATCTGG | |
| | AAGAAATTTGAGCTGCTGCCAACCCCCC | |
| | CCCTATCTCCATCCCGCCGTTCCGGTCTC | |
| | TGCTCTCCTAGCTACGTGGCTGTCACTCC | |
| | CTTTTCGCTGCGTGGGGACAACGATGGG | |
| | GGGGGCGGAAGTTTCAGCACTGCGGACC | |
| | AGCTGGAGATGGTGACCGAGCTGCTGGG | |
| | TGGTGACATGGTCAACCAGTCTTTTATCT | |
| | GCGACCCGGATGACGAGACCTTCATCAA | |
| | GAACATCATCATCCAGGACTGTATGTGG | |
| | TCTGGCTTCTCTGCCGCTGCAAAACTGG | |
| | TCTCGGAGAAACTTGCTAGCTACCAGGC | |
| | TGCTCGCAAGGACTCCGGCTCGCCGAAT | |
| | CCAGCTAGGGGACATAGTGTTTGTAGTA | |
| | CCTCCTCGCTCTACCTGCAGGACCTGTCC | |
| | GCAGCGGCCTCTGAGTGTATTGACCCGT | |
| | CCGTGGTGTTCCCCTATCCTCTCAACGAC | |
| | TCGTCTTCCCCCAAGAGCTGCGCCTCCC | |
| | AGGACTCATCTGCGTTCTCCCCCTCCTCC | |
| | GATAGCCTGTTGAGTAGCACAGAGAGCT | |
| | CCCCTCAGGGCTCCCCGGAGCCCCTGGT | |
| | GCTACACGAGGAGACCCCGCCCACCACC | |
| | AGCAGTGACTCAGAAGAGGAGCAGGAG | |
| | GACGAAGAGGAGATCGATGTGGTTTCCG | |
| | TGGAGAAGCGCCAGGCCCCTGGCAAAC | |
| | GCTCCGAATCCGGCTCCCCTTCCGCCGG | |
| | CGGCCACTCCAAGCCCCCGCACAGCCCG | |
| | TTGGTGCTGAAGAGATGTCACGTGTCAA | |
| | CCCACCAGCATAACTACGCGGCGCCTCC | |
| | CTCTACCCGCAAAGACTACCCCGCTGCC | |
| | AAGCGCGTGAAGTTGGATAGCGTCCGGG | |
| | TCTTACGCCAGATTTCTAACAACCGCAA | |
| | GTGCACTTCTCCAAGGTCTTCCGACACG | |
| | GAGGAGAACGTGAAGCGGCGCACCCAC | |
| | AACGTACTGGAGCGCCAGCGTCGTAACG | |
| | AGTTGAAGCGCAGCTTCTTCGCGCTTCG | |
| | GGACCAGATACCTGAGCTCGAGAATAAC | |
| | GAGAAAGCACCAAAGGTAGTCATCCTG | |
| | AAGAAGGCCACGGCCTACATCTTATCAG | |
| | TGCAGGCCGAGGAGCAGAAGCTGATTTC | |
| | AGAGGAGGATCTGCTGCGCAAGCGCCG | |
| | AGAACAGCTGAAGCACAAGCTGGAACA | |
| | ACTCCGCAACTCATGCGCCTGA | |
| GLIS1 for T cells | ATGGCCGAGGCCCGCACTAGCCTCTCAG | 19 |
| (T-GLIS1) | CGCACTGTCGCGGCCCTCTCGCTACGGG | |
| | CCTGCACCCCGACCTGGACCTGCCCGGG | |
| | CGCAGCCTGGCTACCCCGGCCCCCTCCT | |
| | GTTACCTTTTGGGCTCGGAGCCCTCTAG | |
| | CGGCCTGGGCCTACAGCCAGAGACTCAC | |
| | CTTCCCGAAGGCTCCCTGAAGAGGTGTT | |
| | GCGTCCTTGGCTTGCCGCCCACCTCGCCT | |
| | GCCAGCTCCTCCCCATGCGCGTCTTCAG | |
| | ACGTGACCTCCATCATCCGCTCGTCCCA | |
| | GACCTCCCTGGTGACCTGCGTGAACGGG | |
| | CTGCGTTCTCCCCCGCTCACCGGCGACC | |
| | TGGGAGGTCCGAGTAAGCGCGCTCGGCC | |
| | CGGCCCTGCGAGTACAGACTCTCATGAG | |
| | GGCTCGCTGCAGCTGGAAGCGTGTCGCA | |
| | AGGCCTCCTTCCTGAAGCAGGAGCCAGC | |
| | CGACGAGTTCTCTGAGCTGTTCGGACCT | |
| | CACCAGCAGGGTTTGCCCCCTCCTTACC | |
| | CACTCAGCCAGCTCCCCCCTGGGCCCTC | |
| | TTTGGGCGGTTTAGGCCTGGGGTTAGCG | |
| | GGACGTGTGGTGGCTGGCCGCCAGGCCT | |
| | GCCGATGGGTCGATTGTTGTGCCGCTTA | |
| | CGAGCAGCAGGAGGAGCTGGTGCGCCA | |
| | CATTGAAAAGAGCCATATTGACCAGCGC | |
| | AAGGGCGAGGACTTCACCTGCTTTTGGG | |
| | CTGGGTGCGTGCGCCGCTACAAGCCGTT | |
| | CAACGCGAGATACAAGCTGCTGATCCAC | |
| | ATGCGTGTACACTCCGGAGAGAAACCCA | |
| | ACAAGTGCATGTTTGAGGGATGCTCCAA | |
| | GGCGTTCAGCCGCCTGGAGAACTTGAAA | |
| | ATCCACCTGCGCTCACACACCGGTGAGA | |
| | AGCCATACCTGTGCCAACACCCCGGTTG | |
| | TCAGAAGGCGTTCAGCAACAGCTCCGAT | |
| | CGAGCTAAACACCAGCGGACCCATCTTG | |
| | ACACCAAGCCGTACGCATGCCAAATTCC | |
| | AGGGTGCTCCAAGCGTTACACCGACCCG | |
| | TCCAGTCTGCGCAAGCACGTGAAGGCCC | |
| | ATAGTGCGAAGGAGCAGCAAGTGCGGA | |
| | AGAAGCTCCACGCCGGCCCTGACACGGA | |
| | GGCCGATGTGCTGACCGAGTGCCTGGTT | |
| | CTGCAGCAGCTGCACACTAGCACCCAGC | |
| | TGGCAGCCAGCGATGGCAAAGGTGGTTG | |
| | CGGGCTGGGACAGGAGTTACTGCCAGGC | |
| | GTGTACCCCGGAAGCATCACCCCGCACA | |
| | ACGGCCTAGCCTCTGGACTTCTCCCTCCT | |
| | GCCCATGACGTGCCCTCTCGGCACCACC | |
| | CGCTCGATGCTACCACCTCCTCTCACCA | |
| | CCACCTGTCCCCCTTGCCTATGGCCGAG | |
| | TCCACCCGCGACGGCCTGGGCCCTGGCT | |
| | TGTTGTCGCCCATCGTGTCCCCTCTGAAA | |
| | GGCCTTGGCCCCCCGCCGCTGCCCCCCT | |
| | CTTCTCAGTCCCATAGTCCCGGTGGGCA | |
| | GCCGTTTCCCACCCTGCCCTCTAAGCCCT | |
| | CCTATCCCCCGTTCCAGAGTCCACCCCC | |
| | CCCCCCACTGCCGTCCCCGCAGGGCTAC | |
| | CAGGGCTCCTTCCATTCTATCCAGTCTTG | |
| | CTTTCCTTACGGCGATTGTTACAGGATG | |
| | GCTGAGCCCGCAGCTGGGGGCGATGGG | |
| | CTGGTGGGCGAGACACATGGATTCAATC | |
| | CACTCAGGCCCAACGGTTATCACTCGCT | |
| | ATCCACTCCGTTGCCTGCCACGGGTTAT | |
| | GAGGCGCTGGCAGAGGCTAGCTGCCCA | |
| | ACGGCTCTGCCTCAGCAGCCATCAGAGG | |
| | ACGTGGTCAGCAGCGGCCCTGAAGACTG | |
| | CGGCTTCTTTCCTAATGGGGCCTTCGACC | |
| | ACTGTCTTGGCCACATCCCGTCCATCTAC | |
| | ACAGACACCTGA | |

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the topical compositions and all measurements made are at about 25° C., unless otherwise specified.

As used herein, the term “cell” refers to an intact live cell, naturally occurring or modified. The cell may be isolated from other cells, mixed with other cells in a culture, or within a tissue (partial or intact), or an organism. The methods described herein can be performed, for example, on a sample comprising a single cell, a population of cells, or a tissue or organ comprising cells.

As used herein, the term “cellular reprogramming factors” refers to a set of transcription factors, and combinations thereof, that can convert adult or differentiated cells into pluripotent stem cells. Exemplary reprogramming factors include OCT4, SOX2, KLF4, c-MYC, LIN28, NANOG and/or GLIS1. Other exemplary reprogramming factors include CMYC, DPPA2, DPPA4, ESRRB, GDF3, GLIS1, KLF2, KLF4, KLF5, LIN28, LMYC, NANOG, NMYC, NR5A1, NR5A2, OCT-4, RCOR2, SALL1, SALL4, SOX1, SOX2, SOX3, TDRD12, TET1, TH2A, TH2B, UTF1, ZFP42, MDM2, CyclinD1, SV40 large T antigen, SIRT6, TCL1A, and RARy.

As used herein, the term “mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. In some embodiments, the cell has been genetically engineered to express a recombinant protein and/or nucleic acid.

As used herein, the term “non-integrative” with reference to a messenger RNA (mRNA) refers to an mRNA molecule that is not integrated intrachromosomally or extrachromosomally into the host genome.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “rejuvenated cell(s)” refers to aged cells that have been treated or transiently reprogrammed with one or more cellular reprogramming factors such that the cells have a transcriptomic profile of a younger cell while still retaining one or more cell identity markers.

As used herein, the term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

As used herein, the term “transfection” refers to the uptake of exogenous DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3.sup.rd edition, Cold Spring Harbor Laboratories, New York, Davis et al. (1995) Basic Methods in Molecular Biology, 2.sup.nd edition, McGraw-Hill, and Chu et al. (1981) Gene 13:197. Such techniques can be used to introduce one or more exogenous DNA or RNA molecules into cells. The term refers to both stable and transient uptake of the DNA or RNA molecules. For example, transfection can be used for transient uptake of mRNAs encoding cellular reprogramming factors into cells in need of rejuvenation.

As used herein, the term “transient reprogramming” refers to exposure of cells to cellular reprogramming factors for a period of time sufficient to rejuvenate cells (i.e., eliminate all or some hallmarks of aging), but not long enough to cause dedifferentiation into stem cells. Such transient reprogramming results in rejuvenated cells that retain their identity (i.e., differentiated cell-type).

The term “treating” is used herein, for instance, in reference to methods of treating a cell, a tissue or a subject, and generally includes the administration of a compound or composition which reduces the frequency of, or delays the onset of, symptoms of aging or of a medical condition in a subject relative to a subject not receiving the compound or composition. This can include reversing, reducing, or arresting the symptoms, clinical signs, and underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Cellular and Tissue Treatment

In embodiments, the methods provided herein may be applied to any type of cell in need of rejuvenation. The cell may be isolated from other cells, mixed with other cells in a culture, or within a tissue (partial or intact), or a live organism. The methods described herein can be performed, for example, on a sample comprising a single cell, a population of cells, or a tissue or organ comprising cells. The cells chosen for rejuvenation will depend on the desired therapeutic effect for treating an age-related disease or condition.

In embodiments, the cells are mammalian cells. In embodiments, the cells are human cells. In embodiments, the cells are from an elderly subject.

In embodiments, the methods provided herein may be performed on cells, tissue, or organs of the nervous system, muscular system, respiratory system, cardiovascular system, skeletal system, reproductive system, integumentary system, lymphatic system, excretory system, endocrine system (e.g., endocrine and exocrine), or digestive system. Any type of cell can potentially be rejuvenated, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

In some embodiments, the cells are selected from fibroblasts, endothelial cells, chondrocytes, skeletal muscle stem cells, keratinocytes, mesenchymal stem cells and corneal epithelial cells. In embodiments, the cells are fibroblasts. In embodiments, the cells are endothelial cells. In embodiments, the cells are chondrocytes. In embodiments, the cells are skeletal muscle stem cells. In embodiments, the cells are keratinocytes. In embodiments, the cells are mesenchymal stem cells. In embodiments, the cells are corneal epithelial cells.

In embodiments, the rejuvenated fibroblasts exhibit a transcriptomic profile similar to a transcriptomic profile of young fibroblasts. In embodiments, the rejuvenated fibroblasts exhibit an increased gene expression of one or more nuclear and/or epigenetic markers compared to a reference value as described above. In embodiments, the rejuvenated fibroblasts have a proteolytic activity that is more similar to the proteolytic activity of young cells as described above. In embodiments, the rejuvenated fibroblasts exhibit improved mitochondria health and function compared to a reference value as described above. In embodiments, the rejuvenated fibroblasts exhibit a reversal of the methylation landscape.

In embodiments, the rejuvenated endothelial cells exhibit a transcriptomic profile similar to a transcriptomic profile of young endothelial cells. In embodiments, the rejuvenated endothelial cells exhibit increased gene expression of one or more nuclear and/or epigenetic markers compared to a reference value as described above. In embodiments, the rejuvenated endothelial cells have a proteolytic activity that is more similar to the proteolytic activity of young cells as described above. In embodiments, the rejuvenated endothelial cells exhibit improved mitochondria health and function compared to a reference value as described above. In embodiments, the rejuvenated endothelial cells exhibit a reversal of the methylation landscape.

In embodiments, the rejuvenated chondrocytes exhibit reduced expression of inflammatory factors and/or and increased ATP and collagen metabolism. In embodiments, the inflammatory factors include RANKL, iNOS2, IL6, IFNα, MCP3 and MIP1A. In embodiments, the rejuvenated chondrocytes exhibit reduced expression of RANKL. In embodiments, the rejuvenated chondrocytes exhibit reduced expression of iNOS2. In embodiments, the rejuvenated chondrocytes exhibit reduced expression of IL6. In embodiments, the rejuvenated chondrocytes exhibit reduced expression of IFNα. In embodiments, the rejuvenated chondrocytes exhibit reduced expression of MCP3. In embodiments, the rejuvenated chondrocytes exhibit reduced expression of MIP1A. In embodiments, the rejuvenated chondrocytes exhibit reduced expression of RANKL, iNOS2, IL6, IFNα, MCP3 and MIP1A. In embodiments, the rejuvenated chondrocytes exhibit increased ATP and collagen metabolism. In embodiments, ATP and collagen metabolism is measured by one or more of increased ATP levels, decreased ROS and increased SOD2 expression, increased COL2A1 expression and overall proliferation by the chondrocytes. In embodiments, ATP and collagen metabolism is measured by increased ATP levels. In embodiments, ATP and collagen metabolism is measured by decreased ROS and increased SOD2 expression. In embodiments, ATP and collagen metabolism is measured by increased COL2A1 expression and overall proliferation by the chondrocytes.

In embodiments, the rejuvenated skeletal muscle stem cells exhibit higher proliferative capacity, enhanced ability to differentiate into myoblasts and muscle fibers, restored lower kinetics of activation from quiescence, ability to rejuvenate the muscular microniche, restore youthful force in the muscle, or a combination thereof.

In embodiments, the rejuvenated keratinocytes exhibit higher proliferative capacity, reduced inflammatory phenotype, lower RNAKL and INOS2 expression, reduced expression of cytokines MIP1A, IL6, IFNα, MCP3, increased ATP, increased levels of SOD2 and COL2A1 expression.

In embodiments, the rejuvenated mesenchymal stem cells exhibit reduction in senescence parameters, increased cell proliferation, and/or a decrease in ROS levels. In embodiments, the rejuvenated mesenchymal stem cells exhibit reduction in senescence parameters. In embodiments, the senescence parameters include p16 expression, p21 expression and positive SAβGal staining. In embodiments, the rejuvenated mesenchymal stem cells exhibit increased cell proliferation. In embodiments, the rejuvenated mesenchymal stem cells exhibit a decrease in ROS levels. In embodiments, the rejuvenated mesenchymal stem cells exhibit reduction in senescence parameters, increased cell proliferation, and a decrease in ROS levels.

In embodiments, the rejuvenated corneal epithelial cells exhibit a reduction in senescence parameters. In embodiments, the senescence parameters include one or more of expression of p21, expression of p16, mitochondria biogenesis PGC1α, and expression of inflammatory factor IL8. In embodiments, the senescence parameters include p21. In embodiments, the senescence parameters include expression of p16. In embodiments, the senescence parameters include mitochondria biogenesis PGC1α. In embodiments, the senescence parameters include expression of inflammatory factor IL8. In embodiments, the senescence parameters include one expression of p21, expression of p16, mitochondria biogenesis PGC1α, and expression of inflammatory factor IL8.

The methods of the disclosure can be used to rejuvenate cells in culture (e.g., ex vivo or in vitro) to improve function and potency for use in cell therapy. The cells used in treatment of a patient may be autologous or allogeneic. Preferably, the cells are derived from the patient or a matched donor. For example, in ex vivo therapy, cells are obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, cells in need of rejuvenation can be transfected directly in vivo with mRNAs encoding cellular reprogramming factors.

In another aspect, a method for inducing proliferation of a cell, such as an immune cell, is provided. In some embodiments, the method comprises exposing the cell to mRNA encoding one or more reprogramming factors, whereby said exposing achieves expression of the one or more reprogramming factors in the cell to enhance the proliferation of the cell, with retention of its identity. In some embodiments, the method for inducing proliferation does not induce exhaustion. In some embodiments, the proliferation results from prevention or reversal of exhaustion.

In another aspect, a method for inducing proliferation is performed before, concurrently, or after a method for inhibiting, preventing, or reversing exhaustion. In some embodiments, a method for inducing proliferation is performed at any time understood by one skilled in the art to provide sufficient proliferation before a method for inhibiting, preventing, or reversing exhaustion. In some embodiments, a method for inducing proliferation is performed at any time understood by one skilled in the art to provide sufficient proliferation after a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days before a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days after a method for inhibiting, preventing, or reversing exhaustion.

In some embodiments, methods of the present technology comprise immune cells that are lymphocytes, granulocytes, monocytes, macrophages, microglia, or dendritic cells. In some embodiments, the lymphocyte is a T-cell, a B-cell or a natural killer (NK) cell. In some embodiments, the lymphocyte is a tumor-infiltrating lymphocyte.

In other embodiments, the lymphocyte is a T-cell. In some embodiments, the T-cell is a cytotoxic T cell (CD8+), a helper T cell (CD4+), a suppressor or regulatory T cell (Treg), a memory T cell, a natural killer T cell (NKT cell), or a gamma delta T cell. In other embodiments, the helper T cell is a Th1, T2, Th17, Th9, or Tfh T-cell. In some embodiments, the memory T cell is a central memory T cell, an effector memory T cell, a tissue resident memory T cell, or a virtual memory T cell. In some embodiments, suppressor or regulatory T cells of the present technology are FOXP3+ T cells or FOXP3− T cells. In some embodiments, the NKT cell is a subset of CD1d-restricted T cells.

In some embodiments, a granulocyte of the present technology is a neutrophil, an eosinophil, a basophil, or a mast cell.

In other embodiments, a lymphocyte of the present technology is a B-cell. In some embodiments, a B-cell is a memory B-cell or a plasma cell.

In other embodiments, the immune cell is a monocyte, macrophage, microglial cell, or dendritic cell.

In some embodiments, the methods described herein may be used wherein the immune cell is a natural immune cell or an engineered immune cell. In some embodiments, the methods described herein are performed in parallel or in series with methods of engineering immune cells such that the methods are performed before, during, and/or after the engineering of the immune cells. In some embodiments, such engineering includes engineering so that the immune cells express chimeric antigen receptors. In some embodiments, such chimeric antigen receptors target CD19, CD30, CD33, CD123, FLT3, BCMA, GD2, or any other antigen suitable for immunotherapy. In some embodiments, such engineering includes engineering so that the immune cells express other proteins or peptides, such as growth factors and cytokines. In some embodiments, said cytokines include IL-15. In some embodiments, such engineering of immune cells is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of immune cells is performed ex vivo, e.g., in so-called “in situ” generation of CAR-engineered cells. In such embodiments, mRNA encoding CARs or growth factors or cytokines is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the mRNA encoding the reprogramming factor or factors is delivered in vivo using lipid and/or polymer nanoparticles. In some embodiments, the lipid and/or polymer nanoparticles are engineered for targeted delivery to immune cells in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

In some embodiments, exposing comprises providing a composition comprising the mRNA, wherein the composition comprises an excipient for transfection. In some embodiments, said composition comprises a lipid and the mRNA are associated with the lipid. In some embodiments, the lipids comprise ionizable lipids that can be used in combination with other lipid components, such as helper lipids, stabilization lipids and structural lipids. In some embodiments, the disclosure also provides lipid-nanoparticle compositions comprising such lipids towards delivery of therapeutic nucleic acids. In other embodiments, the composition comprises a polymer and the mRNA are associated with the polymer. In some embodiments, the polymer is a charge-altering releasable transporter. In some embodiments, the charge-altering releasable transporter is at least one of the “block CARTs” or “stat CARTs” described in McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In some embodiments, the polymer or lipid forms a nanoparticle. In other embodiments, said composition comprises both a polymer and lipid and the mRNA are associated with the polymer and/or the lipid. In some embodiments, the use of a lipid or polymer for delivery of the mRNA, such as in a lipid nanoparticle, polymer nanoparticle, or hybrid lipid-polymer nanoparticle, results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA. In some embodiments, the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA due to lower toxicity and/or lower physiological impact on the cell when compared to the different delivery mechanism. In some embodiments, the different delivery mechanism is electroporation such that the use of a lipid or polymer, including lipid or polymer nanoparticles, for delivery of the mRNA results in enhanced rejuvenation, proliferation, recovery from or prevention of exhaustion, anti-pathogenic effects, anti-cancer effects, or anti-inflammatory effects in the exposed immune cell compared to when using electroporation. This improvement compared to electroporation can result from reduced toxicity or reduced physiological impact on the cell compared to electroporation.

In embodiments, the lipids together with the mRNA form a lipid-nanoparticle composition. The lipid-nanoparticle composition can further comprise a helper lipid, a stabilization lipid, and/or a structural lipid. Suitable ionizable lipids, helper lipids, stabilization lipids, structural lipids are described in, for example, U.S. Publication No. 2011/0117125 and in U.S. Pat. Nos. 8,058,069, 9,364,435, 10,703,789, and 11,028,370, the disclosure of lipids therein incorporated by reference herein.

In embodiments, the lipid-nanoparticle composition comprises a phospholipid, and examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

The lipid-nanoparticle composition in some embodiments may comprise a neutral lipid which is either in an uncharged or neutral zwitterionic form depending on pH. The lipid-nanoparticle composition can also comprise a lipid that is a neutral lipid at physiological pH. Examples include diacylphosphatidylcholine, diacylphosphatidylethanolamine, ceramide, sphingomyelin, cephalin, cholesterol, cerebrosides, and diacylglycerols.

The lipid-nanoparticle composition in some embodiments may comprise an anionic lipid, which refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The lipid-nanoparticle composition, in some embodiments, may comprise a cationic lipid which refers to any of a lipid species that carries a net positive charge at a selected pH, such as physiological pH (e.g., pH of about 7.0). In an embodiment, cationic lipids comprising alkyl chains with multiple sites of unsaturation, e.g., at least two or three sites of unsaturation, are used to form the lipid particles. Cationic lipids and related analogs are described in U.S. Patent Publication Nos. 2011/0117125, 2006/0083780 and 2006/0240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference for disclosure of lipid species. In embodiments, the cationic lipids comprise a protonatable tertiary amine (e.g., pH titratable) head group, C18 alkyl chains, ether linkages between the head group and alkyl chains, and 0 to 3 double bonds. Such lipids include, for example, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA) and 1,2-dilinolenyloxy-N,N-dimethyl-3-aminopropane (DLenDMA).

The lipid-nanoparticle composition in some embodiments may comprise a neutral a structural lipid, such as cholesterol, fecosterol, sitosterol, campesterol, stigmasterol, brassicasterol, ergosterol, tomatidine, tomatine, ursolic acid and/or alpha-tocopherol.

The lipid-nanoparticle composition may also comprise a polyethylene glycol (PEG) or PEG-modified lipid. Non-limiting examples include PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-c-DOMG (PEG modified carbamoyl-1,2-dimyristyloxl-propyl-3-amine), PEG-DMG (PEG modified 1,2-dimyristoyl-sn-glycero-3-methoxypolyethylene glycol), PEG-DLPE (PEG modified 1,2-dilauroyl-sn-glycero-3-phosphoethanolamine), PEG-DMPE (PEG modified 1,2-dimyristoyl-sn-glycero-3-phosphoethanolamine), PEG-DPPC (PEG modified 1,2-dipalmitoyl-sn-glycero-3-phosphocholine), or a PEG-DSPE (PEG modified 1,2-distearoyl-sn-glycero-3-phosphoethanolamine-N-[amino(polyethylene glycol)-2000) lipid.

The lipid-nanoparticle composition in some embodiments, may comprise one or more permeability enhancer molecules, carbohydrates, polymers, surface altering agents, or other components. Carbohydrates may include simple sugars, e.g., glucose and polysaccharides, e.g., glycogen and derivatives and analogs thereof.

In some embodiments, lipid nanoparticles or “LNP” are used for delivering the nucleic acids to the cells. As mentioned above, the LNP can comprise natural lipids or synthetic lipids including conjugated lipids or polymers (e.g., PEGylated lipids). The LNPs can comprise any one or more of neutral lipids, zwitterionic, lipids, ionizable lipids, cationic lipids, and anionic lipids. In embodiments, the LNPs comprise natural or synthetic monoacyl or diacyl forms of phosphatidylcholine (PC), phosphatidylglycerol (PG), phosphatidylserine (PS), phosphatidylethanolamine (PE), and phosphatidic acid (PA), or monoacyl, diacyl, triacyl or tetra acyl forms of cardiolipin. In some embodiments, the LNP is a micelle or an inverted micelle (reverse micelle). In other embodiments, the LNP is a unilamellar liposome or a multilamellar liposome.

The cellular aging process has been postulated to be caused by the loss of both genetic and epigenetic information. Loss of genetic information that contributes to cellular aging is typically in the form of genetic mutations such as substitutions, and deletions in an organism's genome. Loss of or changes in epigenetic information associated with cellular aging can take the form of covalent modifications to DNA, such as 5-methylcytosine (5mC), hydroxymethylcytosine (5hmeC), 5-formylcytosine (fC), and 5-carboxylcytosine (caC) and adenine methylation, and to certain proteins, such as lysine acetylation, lysine and arginine methylation, serine and threonine phosphorylation, and lysine ubiquitination and sumoylation of histone proteins. Loss of and changes in the epigenetic information can result in dysregulation of cellular processes, including processes that maintain cell identity, causing cells to exhibit traits that are associated with aging such as senescence.

The methods, compositions, and kits of the present disclosure rejuvenate cells by preventing and reversing the cellular causes of aging. The methods, compositions and kits of the present disclosure rejuvenate cells by restoring epigenetic information that has been lost due to the aging process, injury or disease, as described, for example, in WO2019178296, incorporated by reference herein in its entirety.

The methods for rejuvenating cells provided herein include transfecting cells with one or more non-integrative messenger RNAs encoding one or more cellular reprogramming factors, thereby producing rejuvenated cells. In embodiments, the cells are contacted with, exposed to, or transferred with the mRNA for not more than about 7 days, 6 days, 5 days or 4 days. In embodiments, the rejuvenated cells have a phenotype or activity profile similar to a young cell. The phenotype or activity profile includes one or more of the transcriptomic profile, gene expression of one or more nuclear and/or epigenetic markers, proteolytic activity, mitochondrial health and function. SASP cytokine expression, and methylation landscape.

In some embodiments, the rejuvenated cells have a transcriptomic profile that is more similar to the transcriptomic profile of young cells. In embodiments, the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elf1, Phf8, Pol2s2, Tafi1 and Sin3a.

In some embodiments, the rejuvenated cells exhibit increased gene expression of one or more nuclear and/or epigenetic markers compared to a reference value. In embodiments, the one or more nuclear and/or epigenetic markers is selected from HP1gamma, H3K9me3, lamina support protein LAP2alpha, and SIRT1 protein. In embodiments, the rejuvenated cells have a proteolytic activity that is more similar to the proteolytic activity of young cells. In embodiments, the proteolytic activity is measured as increased cell autophagosome formation, increased chymotrypsin-like proteasome activity, or a combination thereof. In embodiments, the rejuvenated cells exhibit improved mitochondria health and function compared to a reference value. In embodiments, improved mitochondria health and function is measured as increased mitochondria membrane potential, decreased reactive oxygen species (ROS), or a combination thereof.

In some embodiments, the rejuvenated cells exhibit decreased expression of one or more SASP cytokines compared to a reference value. In embodiments, the one or more SASP cytokines include IL18, ILIA, GROA, IL22, and IL9. In embodiments, the rejuvenated cells exhibit reversal of the methylation landscape. In embodiments, the reversal of the methylation landscape is measured by Horvath clock estimation. In some embodiments, a reference value is obtained from an aged cell.

In embodiments, cells are rejuvenated by transient reprogramming with mRNAs encoding one or more cellular reprogramming factors. Transient reprogramming is accomplished, in some embodiments, by transfecting cells once daily with non-integrative mRNAs for at least about two days and not more than about 14, 12, 10, 9, 8, 7, 6, 5, 4 or 3 days. In embodiments, transient reprogramming of cells eliminates various hallmarks of aging while avoiding complete dedifferentiation of the cells into stem cells.

In embodiments, transfecting cells with messenger RNAs may be accomplished by a transfection method, including but not limited to non-viral techniques such as Lipofectamine transfection reagent, LT-1 mediated transfection, dextran-mediated transfection, calcium phosphate precipitation, polybrene-mediated transfection, electroporation, encapsulation of the mRNAs in liposomes, lipid-nanoparticle compositions, and/or direct microinjection.

In an embodiment, the RNA vectors provided herein encode for expression of a combination of one, two, three, four, five, six, or more, reprogramming factors. In an embodiment, the reprogramming factors are selected from Oct4, Klf4, Sox2, c-Myc (or L-myc), Lin28, and Nanog. In an embodiment, the reprogramming factors are Oct4, Klf4, Sox2, c-Myc (or L-myc), Lin28 and Nanog. In an embodiment, the reprogramming factors are Oct4, Klf4, Sox2, c-Myc (or L-myc). In an embodiment, the reprogramming factors are Oct4, Klf4, Sox2. In an embodiment, the reprogramming factors are Oct4, Sox2, Lin28 and Nanog.

In embodiments, a pMK expression vector (Life Technologies), containing a polynucleotide sequence of SEQ ID NOs: 1, a polynucleotide sequence of SEQ ID NO: 2, a polynucleotide sequence of SEQ ID NO: 4, an additionally added internal ribosome entry site (IRES)-GFP, 5′ and 3′ UTRs, and linker regions, is provided for expression and generation of corresponding RNA vectors and/or expression of reprogramming factors as described herein.

In embodiments, a pMK expression vector (Life Technologies), containing a polynucleotide sequence of SEQ ID NOs: 5, a polynucleotide sequence of SEQ ID NO: 6, a polynucleotide sequence of SEQ ID NO: 3, an additionally added internal ribosome entry site (IRES)-GFP, 5′ and 3′ UTRs, and linker regions, is provided for expression and generation of corresponding RNA vectors and/or expression of reprogramming factors as described herein.

In embodiments, a T7-VEE-OKS-iM plasmid, as described in PCT/US2013/041980, containing sequences encoding the non-structural proteins (nsP1 to nsP4) for self-replication, the reprogramming factors Oct4, Klf4, Sox2, and cMyc and an additionally added internal ribosome entry site (IRES)-GFP, is provided for expression and generation of corresponding RNA vectors and/or expression of reprogramming factors as described herein.

In embodiments, self-amplifying RNA molecules are provided, wherein the self-amplifying RNA molecules encode reprogramming factors, such as OCT4 (O), SOX2 (S), KLF4 (K), c-MYC (M), LIN28 (L), NANOG (N), and GLIS1 (G) (each molecule encoding a single factor), that are synthesized via in vitro transcription from plasmid DNA and purified. In embodiments, self-amplifying RNA molecules contain 5′ cap, 5′-UTR, alphavirus NSP1-4 genes, a 26 subgenomic promoter, a coding sequence for a reprogramming factor, a 3′ UTR, and a polyA tail. In other conditions, any individual coding sequence and/or any combination selected from O, S, K, L, M, N and G may be included in the self-amplifying RNA. The alphavirus NSP1-4 genes drive intracellular replication of the self-amplifying RNA after transfection. In embodiments, self-amplifying RNA molecules coding different reprogramming factors are mixed to provide an OSKM cocktail, a OSK cocktail, a OSKG cocktail, a OSKMLN cocktail, or cocktails with other combinations of reprogramming factors (see abbreviations above). In embodiments, the reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). Such self-amplifying RNA molecules and vectors provide advantages over other standard RNA molecules and vectors.

In embodiments, mRNA molecules encoding the reprogramming factors OCT4 (O), SOX2 (S), KLF4 (K), c-MYC (M), LIN28 (L), NANOG (N), and GLIS1 (G) (each molecule encoding a single factor) as well as mRNA molecules encoding B18R are synthesized via in vitro transcription from plasmid DNA and purified. Each mRNA molecule contains a 5′ cap, 5′-UTR, a coding sequence for a single reprogramming factor or B18R, a 3′ UTR, and a polyA tail. Inclusion of mRNA molecules and vectors encoding B18R provide advantages over other standard RNA expression approaches.

In embodiments, monocistronic self-amplifying RNA molecules encoding the reprogramming factors OCT4 (O), SOX2 (S), KLF4 (K), c-MYC (M), LIN28 (L), NANOG (N), and GLIS1 (G) (each molecule encoding a single factor) are provided, wherein each monocistronic mRNA molecule contains a 5′ cap, a 5′-UTR containing L7Ae regulatory sequence, a coding sequence for a single reprogramming factor, a 3′ UTR, and a polyA tail. In other conditions, polycistronic RNA molecules that each encode more than one factor are used. Such vectors including L7Ae on-off switch mechanisms allow control of expression of the reprograming factors and the ability to “shut off” expression at desired time points, providing advantages in control of expression when compared to standard vectors.

In embodiments, polycistronic RNA molecules encoding the reprogramming factors OCT4 (O), SOX2 (S), KLF4 (K), c-MYC (M), LIN28 (L), NANOG (N), and GLIS1 (G) (each molecule encoding two, three, four, five, or six factors, for example LMK and OSK) are provided wherein each mRNA molecule contains a 5′cap, 5′-UTR, coding sequences for two, three, four, five, or six factors, an IRES element or 2A element before each coding sequence such that each gene has its own IRES or 2A element, a 3′ UTR, and a polyA tail. Polycistronic RNA expression increases the likelihood of all reprogramming factors, or the minimum amount of factors required for effective epigenetic reprogramming, to be present in the same cell, and therefore providing advantages over compared to standard vectors.

In embodiments, circular RNA molecules encoding the reprogramming factors OCT4 (O), SOX2 (S), KLF4 (K), c-MYC (M), LIN28 (L), NANOG (N), and GLIS1 (G) (each molecule encoding a single reprogramming factor) are provided via in vitro transcription from plasmid DNA, circularized, and purified. In embodiments, circular RNA molecules are produced using the Anabena intron-exon splicing strategy which consists of a fused partial intron at one end of the RNA and a partial exon at the other end RNA. In embodiments, use of circular RNA allows fewer transfections to be applied and lower RNA doses to be used when compared to conventional mRNA because of the persistence and lower immunogenicity of the circular RNA.

Cellular age-reversal, or rejuvenating, is achieved by transient overexpression of one or more mRNAs encoding cellular reprogramming factors. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In embodiments, the cellular reprogramming factors include one or more of OCT4, SOX2, KLF4, c-MYC, LIN28 and NANOG. In embodiments, the cellular reprogramming factors are applied in different molar ratios, for example OCT4, SOX2, KLF4, c-MYC, LIN28, and NANOG at molar ratios of a:b:c:d:e:f, wherein a, b, c, d, e, and f can all be the same number (for example, 1:1:1:1:1:1), some the same number and some different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or all different numbers (for example 6:4:5:3:2:1), and wherein a, b, c, d, e, and f are each 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors).

In embodiments, the methods provided herein may be applied to any type of cell, tissue or organs in need of rejuvenation. The methods of the disclosure can be used to rejuvenate cells in culture (e.g., ex vivo or in vitro) to improve function and potency for use in cell therapy. The cells used in treatment of a patient may be autologous or allogeneic. Preferably, the cells are derived from the patient or a matched donor. For example, in ex vivo therapy, cells are obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, cells in need of rejuvenation can be transfected directly in vivo with mRNAs encoding cellular reprogramming factors.

### Exemplary Compositions

In an aspect, provided herein are pharmaceutical compositions including rejuvenated cells obtained by transfecting cells with one or more non-integrative messenger RNAs encoding one or more cellular reprogramming factors for not more than 4, 5, 6, or 7 continuous days, to transiently reprogram the cells for rejuvenation.

The compositions provided herein comprise reprogramming factor polynucleotides, proteins, polypeptides and RNA vectors containing said reprogramming factor polynucleotides for production of said reprogramming factor proteins and polypeptides. In some embodiments, the reprogramming factors provided herein provide more robust cellular rejuvenation because the reprogramming factors have been optimized to decrease any triggered immune response to the protein/polypeptide, increase stability of the protein/polypeptide, and altered protein/polypeptide activity, such as increased activity when compared to wild-type reprogramming factors.

In some embodiments, the polynucleotides and corresponding proteins/polypeptides provided herein comprise reprogramming factors such as improved and/or optimized reprogramming factors from the Oct, Sox, Klf, Lin, Nanog, or Myc families that trigger a reduced immune response, are more stable and elicit a more desirable activity, when compared to wild-type reprogramming factors.

The compositions provided herein provide, for example, polynucleotides and corresponding proteins/polypeptides for OCT4, SOX2, c-MYC, KLF4, LIN28 and/or NANOG that trigger a reduced host immune response, are more stable, and exhibit superior activity when compared to wild-type OCT4, SOX2, KLF4, c-MYC, LIN28 and/or NANOG. In some embodiments, the described reprogramming factors comprise altered polynucleotide and/or protein/polypeptide sequences as compared to wild-type reprogramming factors. In some embodiments, the altered polynucleotide and protein/polypeptide sequences provide altered secondary and/or tertiary protein/polypeptide structures when compared to the wild-type structures. These altered secondary and tertiary structures contribute, in some embodiments, to decreased triggered immune response, increased stability, and superior activity of the reprogramming factors described herein.

In some embodiments, the compositions provided herein provide improved OCT4, SOX2, c-Myc, KLF4, LIN28, NANOG, and GLIS1 reprogramming factor polypeptides that are encoded by optimized polynucleotide sequences of SEQ ID NOs: 1-6 and 10, respectively. Accordingly, the provided sequences (SEQ ID NOs: 1-6 and 10) constitute altered polynucleotide sequences when compared to wild-type OCT4, SOX2, c-Myc, KLF4, LIN28, NANOG, and GLIS1. The altered nucleotide sequences (SEQ ID NOs: 1-6 and 10) encode more robust reprogramming factors that elicit a smaller triggered immune response, are more stable and/or provide a more desirable activity level, when compared to polypeptides corresponding to wild-type nucleotide sequences.

The compositions comprise one or more of the RNA vectors described herein, in an embodiment. The RNA vector in the composition can be a bicistronic vector or polycistronic vector that comprises, respectively, two or more, polynucleotide sequences encoding for one or more reprogramming factors. In an embodiment, the polynucleotide sequence in the vector is one having at least about 80%, 81%, 82%, 83%, 84%, 85%, 86%, 87%, 88%, 89%, 90%, 91%, 92%, 93%, 94%, 95%, 96%, 97%, 98%, 99% sequence identity to any one of SEQ ID NOs: 1-6 or 10. By way of example, an RNA vector can comprise from 5′ to 3′: (a promoter)-(a first polynucleotide sequence for a first reprogramming factor)-(a first reprogramming factor separating region)-(a second polynucleotide sequence for a second reprogramming factor)-(a second reprogramming factor separating region)-(a third polynucleotide sequence for a third reprogramming factor)-(optional additional polynucleotide sequences for optional additional reprogramming factors)-(optional additional separating regions)-(optional selectable marker)-(virus 3′UTR or a tail)-(optional selectable marker)-(optional promoter), where the polynucleotide sequence for the first, second, third, and optional additional reprogramming factor has at least about 80%, 81%, 82%, 83%, 84%, 85%, 86%, 87%, 88%, 89%, 90%, 91%, 92%, 93%, 94%, 95%, 96%, 97%, 98%, 99% sequence identity to any one of SEQ ID NOs: 1-6 or 10.

By way of another example, an RNA vector can comprise from 5′ to 3′: (a promoter)-(a first polynucleotide sequence for a first reprogramming factor)-(an optional first reprogramming factor separating region)-(a second polynucleotide sequence for a second reprogramming factor)-(an optional second reprogramming factor separating region)-(a third polynucleotide sequence for a third reprogramming factor)-[(optional additional separating regions) and (optional additional polynucleotide sequences for optional additional reprogramming factors)]ₙ-(optional additional separating regions)-(optional selectable marker)-(virus 3′UTR or a tail)-(optional selectable marker)-(optional promoter), where the polynucleotide sequence for the first, second, third, and optional additional reprogramming factor has 80%, 81%, 82%, 83%, 84%, 85%, 86%, 87%, 88%, 89%, 90%, 91%, 92%, 93%, 94%, 95%, 96%, 97%, 98%, 99% sequence identity to any one of SEQ ID NOs: 1-6 or 10, where n can be 1, 2, 3 or 4.

By way of another example, an RNA vector can comprise from 5′ to 3′: (an optional promoter)-(a first polynucleotide sequence for a first reprogramming factor)-(an optional first reprogramming factor separating region)-(a second polynucleotide sequence for a second reprogramming factor)-[(optional additional separating regions)-(optional additional polynucleotide sequences for optional additional reprogramming factors)]ₙ-(optional additional separating regions)-(optional selectable marker)-(virus 3′UTR or a tail)-(optional selectable marker)-(optional promoter), where the polynucleotide sequence for the first and second, and for the optional additional reprogramming factor has at least about 80%, 81%, 82%, 83%, 84%, 85%, 86%, 87%, 88%, 89%, 90%, 91%, 92%, 93%, 94%, 95%, 96%, 97%, 98%, 99% sequence identity to any one of SEQ ID NOs: 1-6 or 10, where n can be 1, 2, 3 or 4.

In some embodiments, the reprogramming factor separating regions each consist of one or more of the following: an IRES, a promoter and a self-cleaving peptide. In some embodiments, the 5′ UTR has at least about 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 8. In another embodiment, the 3′ UTR is at least 95% has at least about 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 9.

In some embodiments, reprogramming factor separating regions may include linker sequences selected from various linkers that effect expression levels of the downstream factors. For example, linker sequences, such as IRES and 2A peptide linkers, may be included between cistrons of polycistronic mRNA expression vectors. In some embodiments, various linkers drive stronger or weaker expression of downstream genes. For example, certain mutant IRES sequences elicit varying levels of expression of reporter genes downstream of the various mutated IRES sequences. Also, in some embodiments, cistrons that are located further downstream from the promoter sequence may exhibit weaker expression than cistrons closer to the promoter. Therefore, in some embodiments, various linker sequences (e.g., IRES, 2A, and mutants of the same) may be selected and used in a particular order, between cistrons encoding the reprogramming factors as described herein. Appropriate choice of linker, location and order, in the polycistronic vectors described herein, can be used to modulate, control, harmonize, tune, and/or otherwise effect expression of the various reprogramming factors to allow the relative expression ratio of the factors to be present at desirable levels, such as to be equal (e.g., 1:1:1:1:1:1 for O:S:K:M:L:N) or tuned/adjusted (e.g., 2:1:1:1:1:1 for O:S:K:M:L:N) in any manner deemed appropriate for expression of the factor, thus increasing the reprogramming efficiency and generating desirable and robust reprogramming outcomes for the cells treated in accordance with the methods and compositions provided herein.

In some embodiments, a self-replicating virus backbone (the structural genes being removed), such as an alphavirus, may be used to express the reprogramming factors. It should be appreciated that use of such self-replicating virus backbones requires a reduced number of transfections, preferably 1, 2, 3 or 4, into primary human somatic cells to express the reprogramming factors for a therapeutic effect on the cells. For example, generation of an alphavirus RF-RNA transcript utilizes a SP6 (or T7) in vitro transcription kit that does not require special conditions and thereby, further simplifies the approach for broad use. By expressing the one, two, three, four, five or six reprogramming facts at consistent, high levels over time in the same cell combined with replication of the virus-reprogramming factor RNA construct for a desired number of multiple cell generations, the virus-reprogramming factor RNA construct approach solves both of the major inefficiency problems associated with repeated daily transfections of four individual reprogramming factor mRNAs. The virus-reprogramming factor RNA construct is an ectopic approach that does not utilize a DNA intermediate and therefore, there is no opportunity for integrative mutation that can occur with DNA vector-based approaches. In addition, the approach can be engineered to express alternative reprogramming factor combinations and/or insertion of additional reprogramming factor ORFs into the reprogramming factor-RNA backbone.

In some embodiments, the RNA vectors provided herein also include an RNA replicon comprised of a replicase domain from a virus. Reference to an RNA replicon intends an RNA molecule expressing nonstructural protein genes such that it can direct its own replication (self-replication or amplification). An RNA replicon, in embodiments, comprises, 5′ and 3′ virus replication recognition sequences, coding sequences for virus nonstructural proteins, and optionally a polyadenylation tract. It may additionally contain one or more elements, such as an internal ribosome entry site (IRES) sequence, a core or mini-promoter, and the like, to direct the expression, meaning transcription and translation, of a heterologous RNA sequence. The virus replicon can comprise, in one embodiment, 5′ and 3′ virus replication recognition sequences, coding sequences for a virus nonstructural proteins, optional polyadenylation tract, and one or more of a coding sequence selected from the reprogramming factor, such as those described infra.

In an embodiment, the replicase domain is a positive-stranded RNA virus replicase domain. In positive-strand RNA viruses the components of the replicase complex are translated directly from the genomic RNA. Viral polypeptides not required for RNA replication, which mainly constitute structural proteins, can either also be translated from the genomic RNA or from one or more subgenomic mRNAs transcribed from a negative sense cRNA template, depending on the specific type of virus. Genomes of members of the group using the former expression strategy contain one long open reading frame (ORF), and include flaviviruses and picornaviruses. The RNA with positive polarity (genome orientation) is translated into one polyprotein that is subsequently processed into the viral proteins. Translation of this RNA leads to a polyprotein that is co-translationally and posttranslationally processed by viral and host cellular proteases. Viruses that characterized by the subgenomic RNAs used for expression of part of their genes include togaviruses and caliciviruses, which transcribe one RNA of subgenomic length encoding the structural proteins. Coronaviruses and arteriviruses use multiple subgenomic mRNAs for expression of structural and accessory proteins. The replicase genes of these viruses are located in the 5′ part of the genome upstream of the structural genes. For all of these viruses the subgenomic RNAs are 3′ co-terminal with the genomic RNA. Tews and Meyers, RNA Vaccines: Methods and Protocols, Methods in Molecular Biology, Vol 1449, Chapter 2: 2017.

In an embodiment, the replicase domain is comprised of a non-structural replicase domain from a virus, and in an embodiment, the virus an alpha virus. The RNA replicon is, in an embodiment, an alphavirus replicon RNA comprising at least one non-structural replicase domain from an alphavirus and at least one non-alphavirus heterologous sequence encoding factors for a reprogramming factor that when expressed in a somatic cell rejuvenates the cell and/or induces generation of a pluripotent stem cell. In an embodiment, an alphavirus structural protein/protein(s) refers to one or a combination of the structural proteins encoded by alphaviruses. These are produced by the virus as a polyprotein and are represented generally in the literature as C-E3-E2-6k-E1. E3 and 6k serve as membrane translocation/transport signals for the two glycoproteins, E2 and E1. Thus, use of the term E1 herein can refer to E1, E3-E1, 6k-E1, or E3-6k-E1, and use of the term E2 herein can refer to E2, E3-E2, 6k-E2, or E3-6k-E2. Attenuating mutations can be introduced into any one or more of the alphavirus structural proteins.

In an embodiment, the replicon comprises sequences obtained from an alphavirus selected from the group consisting of Eastern Equine Encephalitis virus (EEE), Venezuelan Equine Encephalitis virus (VEE), Everglades virus, Mucambo virus, Pixuna virus Western Equine Encephalitis virus (WEE), Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus and Buggy Creek virus.

The reprogramming factor is a protein, for example a transcription factor, that plays a role in changing adult or differentiated cells into pluripotent stem cells. The term “reprogramming factor” further includes any analogue molecule that mimics the function of the factor. In embodiments, the reprogramming factor is a factor from the Oct family, the Sox family, the Klf family, the Myc family, Nanog family, Glis family, or Lin family.

“Oct family” refers to the family of octamer (“Oct”) transcription factors which play a crucial role in maintaining pluripotency. POU5F1 (POU domain, class 5, transcription factor 1) also known as Oct3/4 is one representative of Oct family. The absence of Oct3/4 in Oct-3/4+cells, such as blastomeres and embryonic stem cells, leads to spontaneous trophoblast differentiation, and presence of Oct-3/4 thus gives rise to the pluripotency and differentiation potential of embryonic stem cells. Exemplary Oct3/4 proteins are the proteins encoded by the murine Oct3/4 gene (Genbank accession number NM_013633) and the human Oct3/4 gene (Genbank accession number NM_002701). The terms “Oct3/4”, “Oct4,” “OCT4,” “Oct4 protein,” “OCT4 protein” and the like thus refer to any of the naturally-occurring forms of the Octomer 4 transcription factor, or variants thereof that maintain Oct4 transcription factor activity (e.g., within at least 50%, 80%, 90% or 100% activity compared to wild-type Oct4 as measured by methods known in the art). In some embodiments, variants have at least 90% amino acid sequence identity across their whole sequence compared to the naturally occurring Oct4 polypeptide. In other embodiments, the Oct4 protein is the protein as identified by the Genbank reference ADW77327.1.

An Oct reprogramming factor refers to any of the naturally-occurring members of octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include, Oct-1, Oct-2, Oct-3/4, Oct-6, Oct-7, Oct-8, Oct-9, and Oct-11. e.g., Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, or 95% amino acid sequence identity across their whole sequence compared to a naturally occurring Oct polypeptide family member such as to those listed above or such as listed in Genbank accession number NP002692.2 (human Oct4) or NP038661.1 (mouse Oct4). Oct polypeptides (e.g., Oct3/4) can be from human, mouse, rat, bovine, porcine, or other animals.

In some embodiments, the OCT4 reprogramming factor protein/polypeptide provided herein is encoded by optimized polynucleotide sequence of SEQ ID NO: 1. Accordingly, SEQ ID NO: 1 constitutes altered polynucleotide sequences when compared to wild-type OCT4. The altered nucleotide sequences, such as SEQ ID NO: 1, encodes, in some embodiments, a more robust OCT4 reprogramming factor that elicits a smaller triggered immune response, is more stable and/or provides a more desirable activity level when compared to proteins or polypeptides corresponding to wild-type nucleotide sequences. In some embodiments, the OCT4 reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 1. In some embodiments, the OCT4 reprogramming factor protein/polypeptide is encoded by a polynucleotide sequence comprising SEQ ID NO: 1. In some embodiments, the OCT4 reprogramming factor protein/polypeptide is encoded by a polynucleotide sequence consisting essentially of SEQ ID NO: 1. In some embodiments, the OCT4 reprogramming factor protein/polypeptide is encoded by a polynucleotide sequence consisting of SEQ ID NO: 1.

“Sox family” refers to genes that encode for SRY (sex determining region Y)-box 2, also known as SOX2, associated with maintaining pluripotency. Exemplary Sox2 proteins are the proteins encoded by the murine Sox2 gene (Genbank accession number NM_011443) and the human Sox2 gene (Genbank accession number NM_003106). The terms “Sox2,” “SOX2,” “Sox2 protein,” “SOX2 protein” and the like as referred to herein thus includes any of the naturally-occurring forms of the Sox2 transcription factor, or variants thereof that maintain Sox2 transcription factor activity (e.g., within at least 50%, 80%, 90% or 100% activity compared to wild-type Sox2 as measured by methods known in the art). In some embodiments, variants have at least 90% amino acid sequence identity across their whole sequence compared to the naturally occurring Sox2 polypeptide. In other embodiments, the Sox2 protein is the protein as identified by the NCBI reference NP_003097.1.

A Sox reprogramming factor refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, e.g., Dang, D. T., et al., Int. J. Biochem. Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals.

In some embodiments, the SOX2 reprogramming factor protein/polypeptide provided herein is encoded by optimized polynucleotide sequence of SEQ ID NO: 2. Accordingly, SEQ ID NO: 2 constitutes altered polynucleotide sequences when compared to wild-type SOX2. The altered nucleotide sequences, such as SEQ ID NO: 2, encode, in some embodiments, a more robust SOX2 reprogramming factor that elicits a smaller triggered immune response, is more stable and/or provides a more desirable activity level when compared to proteins or polypeptides corresponding to wild-type nucleotide sequences. In some embodiments, the SOX2 reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 2. In some embodiments, the SOX2 reprogramming factor protein/polypeptide is encoded by a polynucleotide sequence comprising SEQ ID NO: 2. In some embodiments, the SOX2 reprogramming factor protein/polypeptide is encoded by a polynucleotide sequence consisting essentially of SEQ ID NO: 2. In some embodiments, the SOX2 reprogramming factor protein/polypeptide is encoded by a polynucleotide sequence consisting of SEQ ID NO: 2.

“Klf family” refers to Kruppel-like factor 4 or “Klf” genes that encode for Klf4 proteins are the proteins encoded by the murine klf4 gene (Genbank accession number NM_010637) and the human klf4 gene (Genbank accession number NM_004235). The terms “KLF4,” “KLF4 protein” and the like as referred to herein thus includes any of the naturally-occurring forms of the KLF4 transcription factor, or variants thereof that maintain KLF4 transcription factor activity (e.g., within at least 50%, 80%, 90% or 100% activity compared to wild-type KLF4 as measured by methods known in the art). In some embodiments, variants have at least 90% amino acid sequence identity across their whole sequence compared to the naturally occurring KLF4 polypeptide. In other embodiments, the KLF4 protein is the protein as identified by the NCBI reference NP_004226.3.

In other embodiments, the Klf reprogramming factor refers to any of the naturally-occurring members of the family of Kruppel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J. & Yang, V. W., Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals.

In some embodiments, the KLF4 reprogramming factor protein/polypeptide provided herein is encoded by optimized polynucleotide sequence of SEQ ID NO: 4. Accordingly, SEQ ID NO: 4 constitutes altered polynucleotide sequences when compared to wild-type KLF4. The altered nucleotide sequences, such as SEQ ID NO: 4, encode, in some embodiments, a more robust KLF4 reprogramming factor that elicits a smaller triggered immune response, is more stable and/or provides a more desirable activity level when compared to proteins or polypeptides corresponding to wild-type nucleotide sequences. In some embodiments, the KLF4 reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 4. In some embodiments, the KLF4 reprogramming factor protein/polypeptide is encoded by a polynucleotide sequence comprising SEQ ID NO: 4. In some embodiments, the KLF4 reprogramming factor protein/polypeptide is encoded by a polynucleotide sequence consisting essentially of SEQ ID NO: 4. In some embodiments, the KLF4 reprogramming factor protein/polypeptide is encoded by a polynucleotide sequence consisting of SEQ ID NO: 4.

Factors of the Myc family refers to factors encoded by myc proto-oncogenes implicated in cancer. Exemplary c-Myc proteins are the proteins encoded by the murine c-myc gene (Genbank accession number NM_010849) and the human c-myc gene (Genbank accession number NM_002467). N-Myc or L-myc was also used as possible reprogramming factor replacing c-Myc. The terms “c-Myc,” C-MYC,” “c-Myc protein”, “C-MYC protein” and the like as referred to herein thus includes any of the naturally-occurring forms of the cMyc transcription factor, or variants thereof that maintain cMyc transcription factor activity (e.g., within at least 50%, 80%, 90% or 100% activity compared to wild-type cMyc as measured by methods known in the art). In some embodiments, variants have at least 90% amino acid sequence identity across their whole sequence compared to the naturally occurring c-Myc polypeptide. In other embodiments, the c-Myc protein is the protein as identified by the NCBI reference NP_002458.2.

The Myc family of cellular genes is comprised of c-myc, N-myc, and L-myc, and reference to Myc refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals.

In some embodiments, the c-Myc reprogramming factor protein/polypeptide provided herein is encoded by optimized polynucleotide sequence of SEQ ID NO: 3. Accordingly, SEQ ID NO: 3 constitutes altered polynucleotide sequences when compared to wild-type c-Myc. The altered nucleotide sequences, such as SEQ ID NO: 3, encode, in some embodiments, a more robust c-Myc reprogramming factor that elicits a smaller triggered immune response, is more stable and/or provides a more desirable activity level when compared to proteins or polypeptides corresponding to wild-type nucleotide sequences. In some embodiments, the c-Myc reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 3. In some embodiments, the c-Myc reprogramming factor protein/polypeptide is encoded by a polynucleotide sequence comprising SEQ ID NO: 3. In some embodiments, the c-Myc reprogramming factor protein/polypeptide is encoded by a polynucleotide sequence consisting essentially of SEQ ID NO: 3. In some embodiments, the c-Myc reprogramming factor protein/polypeptide is encoded by a polynucleotide sequence consisting of SEQ ID NO: 3.

The term “Nanog” or “nanog” refers to a transcription factor critically involved with self-renewal of undifferentiated embryonic stem cells. In humans, this protein is encoded by the NANOG gene. Exemplary nanog is the protein encoded by murine gene (Genbank accession number XM.sub.13 132755) and human Nanog gene (Genbank accession number NM_024865). The term “Nanog” or “nanog” and the like includes any of the naturally-occurring forms of the Nanog transcription factor, or variants thereof that maintain Nanog transcription factor activity (e.g., within at least 50%, 80%, 90% or 100% activity compared to wild-type Nanog as measured by methods known in the art). In some embodiments, variants have at least 90% amino acid sequence identity across their whole sequence compared to the naturally occurring Nanog polypeptide. In other embodiments, the Nanog protein is the protein as identified by the NCBI reference NP_079141.

In some embodiments, the Nanog reprogramming factor protein/polypeptide provided herein is encoded by optimized polynucleotide sequence of SEQ ID NO: 6. Accordingly, SEQ ID NO: 6 constitutes altered polynucleotide sequences when compared to wild-type Nanog. The altered nucleotide sequences, such as SEQ ID NO: 6, encode, in some embodiments, a more robust Nanog reprogramming factor that elicits a smaller triggered immune response, is more stable and/or provides a more desirable activity level when compared to proteins or polypeptides corresponding to wild-type nucleotide sequences. In some embodiments, the Nanog reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 6. In some embodiments, the Nanog reprogramming factor protein/polypeptide is encoded by a polynucleotide sequence comprising SEQ ID NO: 6. In some embodiments, the Nanog reprogramming factor protein/polypeptide is encoded by a polynucleotide sequence consisting essentially of SEQ ID NO: 6. In some embodiments, the Nanog reprogramming factor protein/polypeptide is encoded by a polynucleotide sequence consisting of SEQ ID NO: 6.

The term “Lin28” or “Lin-28 homolog A” is a protein that is encoded by the LIN28 gene in humans. Exemplary Lin28 is the protein encoded by murine gene (Genbank accession number NM_145833) and human Lin28 gene (Genbank accession number NM_024674). The term “Lin28” or “Lin28 homolog A” and the like as referred to herein thus includes any of the naturally-occurring forms of the Lin28 transcription factor, or variants thereof that maintain Lin28 transcription factor activity (e.g., within at least 50%, 80%, 90% or 100% activity compared to wild-type Lin28 as measured by methods known in the art). In some embodiments, variants have at least 90% amino acid sequence identity across their whole sequence compared to the naturally occurring Lin28 polypeptide. In other embodiments, the Lin28 protein is the protein as identified by the NCBI reference NP_078950.

In some embodiments, the Lin28 reprogramming factor protein/polypeptide provided herein is encoded by optimized polynucleotide sequence of SEQ ID NO: 5. Accordingly, SEQ ID NO: 5 constitutes altered polynucleotide sequences when compared to wild-type Lin28. The altered nucleotide sequences, such as SEQ ID NO: 5, encode, in some embodiments, a more robust Lin28 reprogramming factor that elicits a smaller triggered immune response, is more stable and/or provides a more desirable activity level when compared to proteins or polypeptides corresponding to wild-type nucleotide sequences. In some embodiments, the Lin28 reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 5. In some embodiments, the Lin28 reprogramming factor protein/polypeptide is encoded by a polynucleotide sequence comprising SEQ ID NO: 5. In some embodiments, the Lin28 reprogramming factor protein/polypeptide is encoded by a polynucleotide sequence consisting essentially of SEQ ID NO: 5. In some embodiments, the Lin28 reprogramming factor protein/polypeptide is encoded by a polynucleotide sequence consisting of SEQ ID NO: 5.

The terms “Glis”, “GLIS family zinc finger 1” or “Glis1” refer to a protein or protein family that is encoded by the GLIS family of genes, such as the GLIS1 gene in humans (Gene ID: 148979). The term “GLIS family zinc finger 1” or “Glis1” and the like as referred to herein thus includes any of the naturally-occurring forms of the Glis1 transcription factor, or variants thereof that maintain Glis1 transcription factor activity (e.g., within at least 50%, 80%, 90% or 100% activity compared to wild-type Glis1 as measured by methods known in the art). In some embodiments, variants have at least 90% amino acid sequence identity across their whole sequence compared to the naturally occurring Glis1 polypeptide.

Glis1 was identified from a screening of over 1400 transcription factors and is thought to be enriched in unfertilized eggs and embryos at the one cell stage where it can promote direct reprogramming of somatic cells to induced pluripotent stem cells (iPS cells). Glis1 is believed to regulate expression of numerous genes, either positively or negatively, by promoting multiple pro-reprogramming pathways. These pathways are believed to be activated due to the up regulation of the transcription factors N-Myc, Myc11, c-Myc, Nanog, ESRRB, FOXA2, GATA4, NKX2-5, as well as the other factors used for reprogramming. In some embodiments, Glis1 enhances cellular reprogramming and/or rejuvenation when expressed in combination with other reprogramming factors, such as OCT4, SOX2, KLF4 and/or c-MYC. In other embodiments, over expression of Glis1 provides synergistic effects with Nanog in improving reprogramming efficiency. It is believed that Glis1 may interact with Nanog to enhance reprogramming efficiency by stimulating the MET receptor tyrosine kinase and activating the Wingless/Integrated (WNT) signaling pathway.

In some embodiments, the Glis1 reprogramming factor protein/polypeptide provided herein is encoded by optimized polynucleotide sequence of SEQ ID NO: 10. Accordingly, SEQ ID NO: 10 constitutes altered polynucleotide sequences when compared to wild-type Glis1. The altered nucleotide sequences, such as SEQ ID NO: 10, encode, in some embodiments, a more robust Glis1 reprogramming factor that elicits a smaller triggered immune response, is more stable and/or provides a more desirable activity level when compared to proteins or polypeptides corresponding to wild-type nucleotide sequences. In some embodiments, the Glis1 reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 10. In some embodiments, the Glis1 reprogramming factor protein/polypeptide is encoded by a polynucleotide sequence comprising SEQ ID NO: 10. In some embodiments, the Glis1 reprogramming factor protein/polypeptide is encoded by a polynucleotide sequence consisting essentially of SEQ ID NO: 10. In some embodiments, the Glis1 reprogramming factor protein/polypeptide is encoded by a polynucleotide sequence consisting of SEQ ID NO: 10.

In some embodiments, reprogramming factors provided herein comprise T cell optimized factors. In some embodiments, the T cell optimized reprogramming factors protein/polypeptide provided herein are encoded by optimized polynucleotide sequences of SEQ ID NOs: 11-19. Accordingly, SEQ ID NOs: 11-19 constitute altered polynucleotide sequences when compared to wild-type T cell reprogramming factors. The altered nucleotide sequences, such as SEQ ID NOs: 11-19, encode, in some embodiments, a more robust T cell reprogramming factor that elicits a smaller triggered immune response, is more stable and/or provides a more desirable activity level when compared to proteins or polypeptides corresponding to wild-type nucleotide sequences. In some embodiments, the T cell optimized reprogramming factor protein/polypeptide is encoded by a polynucleotide sequence having at least about 80%, about 85%, about 90%, about 95%, about 96%, about 97%, about 98%, about 99%, or about 100% sequence identity to any one of the sequences of SEQ ID NOs: 11-19. In some embodiments, the T cell optimized reprogramming factor protein/polypeptide is encoded by a polynucleotide sequence comprising any one of the sequences of SEQ ID NOs: 11-19. In some embodiments, the T cell optimized reprogramming factor protein/polypeptide is encoded by a polynucleotide sequence consisting essentially of any one of the sequences of SEQ ID NOs: 11-19. In some embodiments, the T cell optimized reprogramming factor protein/polypeptide is encoded by a polynucleotide sequence consisting of any one of the sequences of SEQ ID NOs: 11-19.

In some embodiments, the T cell optimized reprogramming factor comprises OCT4MyoD for T-cells (T-OCT4MyoD, SEQ ID NO: 11) or reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 11. In some embodiments, the T cell optimized reprogramming factor comprises B18R for T cells (T-B18R, SEQ ID NO: 12) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 12. In some embodiments, the T cell optimized reprogramming factor comprises KLF4 for T cells (T-KLF4, SEQ ID NO: 13) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 13. In some embodiments, the T cell optimized reprogramming factor comprises LIN28 for T cells (T-LIN28, SEQ ID NO: 14) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 14. In some embodiments, the T cell optimized reprogramming factor comprises NANOG for T cells (T-NANOG, SEQ ID NO: 15) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 15. In some embodiments, the T cell optimized reprogramming factor comprises OCT4 for T cells (T-OCT4, SEQ ID NO: 16) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 16. In some embodiments, the T cell optimized reprogramming factor comprises SOX2 for T cells (T-SOX2, SEQ ID NO: 17) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 17. In some embodiments, the T cell optimized reprogramming factor comprises cMYC for T-cells (T-cMyc, SEQ ID NO: 18) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 18. In some embodiments, the T cell optimized reprogramming factor comprises GLIS1 for T-cells (T-GLIS1, SEQ ID NO: 19) or a reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 19.

In an embodiment, the RNA vectors provided herein encode for expression of a combination of one, two, three, four, five, six, or more, reprogramming factors. In an embodiment, the reprogramming factors are selected from Oct4, Klf4, Sox2, c-Myc (or L-myc), Lin28 and Nanog.

In an embodiment, the provided RNA vector encodes for expression of OCT4, SOX2, and KLF4 and comprises sequences consisting of SEQ ID NOs: 1, 2 and 4. In an embodiment, the provided RNA vector encodes for expression of LIN28, Nanog and c-Myc and comprises sequences consisting of SEQ ID NOs: 5, 6 and 3. In other embodiments the provided RNA vectors comprise one, two, three, four, five or six polynucleotide sequences that are independently selected from the group consisting of nucleotides comprising SEQ ID NOs: 1-6 and 10.

In other embodiments, the RNA encodes comprises at least two heterologous polynucleotide sequences that encode reprogramming factors. The RNA comprises from 5′ to 3′: (a promoter)-(a first reprogramming factor)-(a first reprogramming factor separating region)-(a second reprogramming factor)-(a second reprogramming factor separating region)-(a third reprogramming factor)-(a third reprogramming factor separating region) (optional additional reprogramming factors-optional additional separating regions)-(optional selectable marker)-(virus 3′UTR and/or polyA or heteropolymer tail)-(optional selectable marker)-(optional promoter). The reprogramming factors are heterologous polynucleotide sequences which encode for a reprogramming factor. As described above, the reprogramming factor can be selected from the group consisting of Oct polypeptides, Klf polypeptides, Sox polypeptides, Myc polypeptides, Nanog, and/or Lin28.

In certain embodiments, compositions comprising rejuvenated cells for use in cell therapy may further comprise one or more additional factors, such as nutrients, cytokines, growth factors, extracellular matrix (ECM) components, antibiotics, anti-oxidants, or immunosuppressive agents to improve cell function or viability. The composition may also further comprise a pharmaceutically acceptable carrier.

Examples of growth factors include, but are not limited to, fibroblast growth factor (FGF), insulin-like growth factor (IGF), transforming growth factor beta (TGF-P), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

Examples of ECM components include, but are not limited to, proteoglycans (e.g., chondroitin sulfate, heparan sulfate, and keratan sulfate), non-proteoglycan polysaccharides (e.g., hyaluronic acid), fibers (e.g., collagen and elastin), and other ECM components (e.g., fibronectin and laminin).

Examples of immunosuppressive agents include, but are not limited to, steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

One or more pharmaceutically acceptable excipients may also be included. Examples include, but are not limited to, carbohydrates, inorganic salts, antimicrobial agents, antioxidants, surfactants, buffers, acids, bases, and combinations thereof.

For example, an antimicrobial agent for preventing or deterring microbial growth may be included. Non-limiting examples of antimicrobial agents suitable for the present disclosure include benzalkonium chloride, benzethonium chloride, benzyl alcohol, cetylpyridinium chloride, chlorobutanol, phenol, phenylethyl alcohol, phenylmercuric nitrate, thimersol, and combinations thereof. Antibmicrobial agents also include antibiotics that can also be used to prevent bacterial infection. Examples antibiotics include amoxicillin, penicillin, sulfa drugs, cephalosporins, erythromycin, streptomycin, gentamicin, tetracycline, chlarithromycin, ciproflozacin, azithromycin, and the like. Also included are antifungal agents such as myconazole and terconazole.

Various antioxidants can also be included, such as molecules having thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hvdroxyanisole (BHA), vitamin K, and the like.

Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate di basic, and combinations thereof.

Acids or bases can also be present as an excipient. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

Methods of Use

The term “age-related disease or condition” refers to any condition, disease, or disorder associated with aging such as, but not limited to, neurodegenerative diseases (e.g., Alzheimer's disease, Parkinson's disease, Huntington's disease, amyotrophic lateral sclerosis, dementia, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), conditions associated with cellular proliferation, and any other diseases and disorders associated with aging.

As used herein, the term “disease or disorder involving cartilage degeneration” is any disease or disorder involving cartilage and/or joint degeneration. The term “disease or disorder involving cartilage degeneration” includes conditions, disorders, syndromes, diseases, and injuries that affect spinal discs or joints (e.g., articular joints) in animals, including humans, and includes, but is not limited to, arthritis, chondrophasia, spondyloarthropathy, ankylosing spondylitis, lupus erythematosus, relapsing polychondritis, and Sjogren's syndrome.

As used herein, the term “muscle degeneration disease or disorder” is any disease or disorder involving muscle degeneration. The term includes conditions, disorders, syndromes, diseases, and injuries that affect muscle tissue such as, but not limited to, muscle atrophy, muscle disuse, muscle tears, burns, surgery, peripheral neuropathy, multiple sclerosis, amyotrophic lateral sclerosis (ALS), Duchenne muscular dystrophy, primary lateral sclerosis, myasthenia gravis, cancer, AIDS, congestive heart failure, chronic obstructive pulmonary disease (COPD), liver disease, renal failure, eating disorders, malnutrition, starvation, infections, or treatment with glucocorticoids.

Conditions associated with cellular proliferation refers to a disease that occurs due to abnormal growth or extension by the multiplication of cells (Walker, Cambridge Dictionary of Biology, Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

“Cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer, anal cancer; angiosarcoma (e.g., lymphangio sarcoma, lymphangioendotheliosarcoma, hemangiosarcoma); appendix cancer, benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer, epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer, gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macro globulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer, inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer, myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer, rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer, sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer, vaginal cancer; and vulvar cancer (e.g., Paget's disease of the vulva).

By “therapeutically effective dose or amount” is intended an amount of rejuvenated cells or intracellular expression of the one or more reprograming factors (including reprogramming factors encoded by SEQ ID NOs: 1-6 and 10) that brings about a positive therapeutic response in a subject in need of tissue repair or regeneration, such as an amount that restores function and/or results in the generation of new tissue at a treatment site. The rejuvenated cells may be produced by transfection in vitro, ex vivo, or in vivo with the RNA, or RNA vector for expression of the one or more reprogramming nucleotide sequences encoding one or more cellular reprogramming factors, as described herein (including SEQ ID NOs: 1-6 and 10). Thus, for example, a “positive therapeutic response” would be an improvement in the age-related disease or condition in association with the therapy, and/or an improvement in one or more symptoms of the age-related disease or condition in association with the therapy, such as restored tissue functionality, reduced pain, improved stamina, increased strength, increased mobility, and/or improved cognitive function. The exact amount (of cells or mRNA) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

For example, a therapeutically effective dose or amount of rejuvenated chondrocytes is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

In another example, a therapeutically effective dose or amount of rejuvenated skeletal muscle stem cells is intended an amount that, when administered as described herein, brings about a positive therapeutic response in a subject having muscle damage or loss, such as an amount that results in the generation of new myofibers at a treatment site (e.g., a damaged muscle). For example, a therapeutically effective dose or amount could be used to treat muscle damage or loss resulting from a traumatic injury or a disease or disorder involving muscle degeneration. Preferably, a therapeutically effective amount improves muscle strength and function.

In some embodiments, the methods of the present technology comprise exposing a cell, such as an immune cell, to RNA for a dosing interval understood by one of ordinary skill in the art to rejuvenate the cell without resulting in a loss of identity or differentiation. In some embodiments, the methods of the present technology comprise exposing a cell to RNA for a dosing interval of not more than 25, 24, 23, 22, 21, 20, 19, 18, 17, 16, 15, 14, 13, 12, 11, 10, 9, 8, 7, 6, 5, 4, or 3 consecutive days. In some embodiments, the RNA dosing, such as mRNA dosing, is performed at least once daily during the dosing interval. In some embodiments, the RNA dosing is performed less frequently than once per day during the dosing interval, for example once every two days, once every three days, once every four days, once every x days, where x is a number from 4 to 25. Thus, in such embodiments, for example, dosing RNA once every 5 days in a 5 day dosing interval means that the RNA is dosed once in the interval, i.e., once in the total treatment period of 5 days, whereas dosing RNA twice daily in a 5 day dosing interval means that the RNA is dosed 10 times in the interval, i.e., 10 times in the 5 days. In some embodiments, the methods of the present technology comprise exposing a cell to RNA for not more than 21, 18, 14, 10, 7, or 5 consecutive days. In some embodiments, the methods of the present technology comprise exposing a cell to RNA for not more than 18 consecutive days. In some embodiments, the methods of the present technology comprise exposing a cell to RNA for not more than 14 consecutive days. In some embodiments, the methods of the present technology comprise exposing a cell to RNA for not more than 10 consecutive days. In some embodiments, the methods of the present technology comprise exposing a cell to RNA at least once daily for not more than 5 consecutive days. In other embodiments, said exposing comprises interrupting said exposing and repeating said exposing after said interrupting. In some embodiments, said exposing comprises exposing the cell to RNA for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive, or between about 19-21 consecutive days and in some embodiments, further comprising interrupting said exposing and repeating said exposing after said interrupting. In some embodiments, the duration of exposure is controlled by the mechanisms described herein, e.g., use of self-amplifying RNA, circular RNA, B18R and other decoys, and/or on/off switches. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time, for example until a disease is successfully treated or cured, or throughout the life of a subject or patient. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One exposure period is considered to be a dosing interval, such that, for example, a sequence of exposure-interruption-repeat exposure contains two dosing intervals.

As used herein, the terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In some cases, the methods of the disclosure find use in experimental animals, in veterinary application, and in the development of animal models for disease. The term does not denote a particular age. Thus, both adult and newborn individuals are intended to be covered.

### Kits

The disclosure also provides kits comprising one or more containers holding compositions comprising one or more non-integrative messenger RN As encoding one or more cellular reprogramming factors for transient reprogramming of cells. Kits may further comprise transfection agents, media for culturing cells, and optionally one or more other factors, such as growth factors, ECM components, antibiotics, and the like. The mRNAs encoding cellular reprogramming factors and/or other compositions can be in liquid form or lyophilized. Such kits may also include components that preserve or maintain the mRNAs that protect against their degradation. Such components may be RNAse-free or protect against RNAses. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating age-related disease or conditions. The package insert can be an unapproved draft package insert or can be a package insert approved by the Food and Drug Administration (FDA) or other regulatory body.

In certain embodiments, the kit comprises mRNAs encoding one or more cellular reprogramming factors selected from the group consisting of OCT4, SOX2, KLF4, c-MYC, LIN28 and NANOG.

### III. Examples

The following examples are illustrative in nature and are in no way intended to be limiting.

### Example 1

### Polycistronic RNA Vector for Expression of Optimized OCT4, SOX2, and KLF4

A pMK expression vector (Life Technologies), containing a polynucleotide sequence of SEQ ID NOs: 1, a polynucleotide sequence of SEQ ID NO: 2, a polynucleotide sequence of SEQ ID NO: 4, an additionally added internal ribosome entry site (IRES)-GFP, 5′ and 3′ UTRs, and linker regions, is amplified in E. coli and plasmids are isolated using QIAPrep (Qiagen, Hilden, Germany). After the linearization, 10 μg template DNA is transcribed in vitro using RiboMAX large-scale production system T7 Kit (Promega, Madison, Wis., USA) according to the manufacturer's instructions. Afterwards, 2 U TURBO DNase is added for 15 min at 37° C. For 5′-end capping, ScriptCap Cap1 Capping System is used followed by 30-end polyadenylation with A-Plus Poly(A) Polymerase Tailing Kit (both from Cellscript, Madison, Wis., USA) according to the manufacturer's instructions. Following each reaction step, RNA is purified using RNeasy Kit (Qiagen). The specific lengths of the generated DNA and RNA products are analyzed using 1% agarose gel electrophoresis.

### Example 2

### Polycistronic RNA Vector for Expression of Optimized LIN28, Nanog, and c-Myc

A pMK expression vector (Life Technologies), containing a polynucleotide sequence of SEQ ID NOs: 5, a polynucleotide sequence of SEQ ID NO: 6, a polynucleotide sequence of SEQ ID NO: 3, an additionally added internal ribosome entry site (IRES)-GFP, 5′ and 3′ UTRs, and linker regions, is amplified in E. coli and plasmids are isolated using QIAPrep (Qiagen, Hilden, Germany). After the linearization, 10 μg template DNA is transcribed in vitro using RiboMAX large-scale production system T7 Kit (Promega, Madison, Wis., USA) according to the manufacturer's instructions. Afterwards, 2 U TURBO DNase is added for 15 min at 37° C. For 5′-end capping, ScriptCap Cap1 Capping System is used followed by 30-end polyadenylation with A-Plus Poly(A) Polymerase Tailing Kit (both from Cellscript, Madison, Wis., USA) according to the manufacturer's instructions. Following each reaction step, RNA is purified using RNeasy Kit (Qiagen). The specific lengths of the generated DNA and RNA products are analyzed using 1% agarose gel electrophoresis.

### Example 3

### Self-Replicating RNA (srRNA)

A T7-VEE-OKS-iM plasmid, as described in PCT/US2013/041980, containing sequences encoding the non-structural proteins (nsP1 to nsP4) for self-replication, the reprogramming factors Oct4, Klf4, Sox2, and cMyc and an additionally added internal ribosome entry site (IRES)-GFP is amplified in E. coli and plasmids are isolated using QIAPrep (Qiagen, Hilden, Germany). After the linearization with MluI restriction enzyme (Thermo Fisher Scientific), 10 μg template DNA is transcribed in vitro using RiboMAX large-scale production system T7 Kit (Promega, Madison, Wis., USA) according to the manufacturer's instructions. Afterwards, 2 U TURBO DNase is added for 15 min at 37° C. For 5′-end capping, ScriptCap Cap1 Capping System is used followed by 30-end polyadenylation with A-Plus Poly(A) Polymerase Tailing Kit (both from Cellscript, Madison, Wis., USA) according to the manufacturer's instructions. Following each reaction step, srRNA is purified using RNeasy Kit (Qiagen). The specific lengths of the generated DNA and srRNA products are analyzed using 1% agarose gel electrophoresis.

### Example 4

### Self-Amplifying RNA for Expression of Reprogramming Factors

Self-amplifying RNA molecules encoding the reprogramming factors OCT4 (O), SOX2 (S), KLF4 (K), c-MYC (M), LIN28 (L), NANOG (N), and GLIS1 (G) (each molecule encoding a single factor) are synthesized via in vitro transcription from plasmid DNA and purified. Each self-amplifying RNA molecule contains a 5′ cap, 5′-UTR, alphavirus NSP1-4 genes, a 26 subgenomic promoter, a coding sequence for a reprogramming factor, a 3′ UTR, and a polyA tail. In other conditions, any individual coding sequence and/or any combination selected from O, S, K, L, M, N and G may be included in the self-amplifying RNA. The alphavirus NSP1-4 genes drive intracellular replication of the self-amplifying RNA after transfection. Self-amplifying RNA molecules coding different reprogramming factors are then mixed to provide an OSKM cocktail, a OSK cocktail, a OSKG cocktail, a OSKMLN cocktail, or cocktails with other combinations of reprogramming factors (see abbreviations above). The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). As a control, conventional mRNA molecules each encoding a single reprogramming factor are also synthesized via in vitro transcription from plasmid DNA, purified, and mixed to form cocktails. Human fibroblasts are obtained from Lonza and cultured in FGM-2 medium. An aging model is induced in the fibroblasts through treatment with TGF-beta at a concentration of 0.1 to 20 ng/ml for 3 days (“aged”, with untreated fibroblasts used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×106 cells/well and allowed to grow to 70% confluency in FGM-2 medium. Self-amplifying RNA molecules are prepared as naked RNA in nuclease-free H2O and then mixed together to provide reprogramming factor combinations of OSKMLN, OSKM, OSK, and OSKG. In other conditions, any individual coding sequence and/or any combination selected from O, S, K, L, M, N and G may be included in the self-amplifying RNA. mRNA molecules are similarly prepared and mixed to provide the same factor combinations. Self-amplifying RNA multifactor cocktails prepared in this manner are mixed with Lipofectamine MessengerMAX at a ratio of 1:1 to form transfection complexes per the manufacturer's instructions. Multifactor mRNA cocktails are similarly mixed with Lipofectamine MessengerMAX to form transfection complexes. The self-amplifying RNA transfection complexes are then added to the wells containing “aged” and/or “control” fibroblasts at doses of 5000 ng RNA per well, and transfection is allowed to proceed for 6 hours. Wells receiving mRNA transfection complexes serve as a control. After transfection is complete, the transfection medium is discarded and fresh medium is applied to the wells. Self-amplifying RNA is transfected once, on Day 1 at the beginning of the experiment. Conventional mRNA is transfected every day.

At 3, 4, 5, 6, and/or 7 days, cell viability and/or proliferation is evaluated using cell proliferation assays (WST-8 or MTT) assay per the manufacturer's instructions (Sigma Aldrich).

At 3, 4, 5, 6, and/or 7 days, cells are stained with specific antibodies and imaged using confocal microscopy to assess the expression of collagen IV, fibronectin and laminin as rejuvenation markers; vimentin as an aging marker; IFIT1, IFIT2, IFIT3, IL6, INFB, OAS1, PKR and TLR3 as cellular immune response markers; and LDH assay and AK assay to measure toxicity.

At 3, 4, 5, 6, and/or 7 days, cells are lysed and total RNA is collected and reverse-transcribed to cDNA. Real-time PCR is used to assess the expression of collagen IV, fibronectin and laminin as rejuvenation markers; vimentin as an aging marker, IFIT1, IFIT2, IFIT3, IL6, INFB, OAS1, PKR and TLR3 as cellular immune response markers; and LDH assay and AK assay to measure toxicity.

Use of self-amplifying RNA allows fewer transfections to be applied and lower RNA doses to be used when compared to conventional mRNA because of the continued propagation of the self-amplifying RNA. Fewer transfections and lower RNA dose will lead to lower toxicity and as a result to higher reprogramming efficacy and stronger cellular rejuvenation effects. Accordingly, self-amplifying RNA improves cell viability and proliferation than conventional mRNA. Thus, treatment with self-amplifying RNA results in up-regulation of cell rejuvenation markers and up-regulation of cell immune response compared to conventional mRNA, while toxicity, and aging markers are down-regulated with self-amplifying RNA compared to treatment with conventional mRNA.

### Example 5

### Co-expression of B18R and Reprogramming Factors

mRNA molecules encoding the reprogramming factors OCT4 (O), SOX2 (S), KLF4 (K), c-MYC (M), LIN28 (L), NANOG (N), and GLIS1 (G) (each molecule encoding a single factor) as well as mRNA molecules encoding B18R are synthesized via in vitro transcription from plasmid DNA and purified. Each mRNA molecule contains a 5′ cap, 5′-UTR, a coding sequence for a single reprogramming factor or B18R, a 3′ UTR, and a polyA tail. Human fibroblasts are obtained from Lonza and cultured in FGM-2 medium. An aging model is induced in the fibroblasts through treatment with TGF-beta at a concentration of 0.1-20 ng/ml for 3 days (“aged”), with untreated fibroblasts used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×106 cells/well and allowed to grow to 70% confluency in FGM-2 medium. mRNA molecules coding different reprogramming factors are prepared as naked RNA in nuclease-free H2O and then mixed together to provide an OSKM cocktail, a OSK cocktail, a OSKG cocktail, a OSKMLN cocktail, or cocktails with other combinations of reprogramming factors (see abbreviations above). The reprogramming factor cocktails contain the reprogramming factor-coding mRNAs in identical proportions (e.g., 1:1:1:1:1:1 for O:S:K:L:M:N) or with proportions of individual factors adjusted (e.g., 2:1:1:1:1:1 for O:S:K:L:M:N). When the cocktails are prepared, mRNA encoding B18R is added to provide combinations such as: OSKMLN+B18R, OSKM+B18R, OSK+B18R, and OSKG+B18R. As a control, combinations of mRNA encoding reprogramming factors without mRNA encoding B18R, e.g., OSKMLN, OSKM, OSK, and OSKG, are used. mRNA cocktails prepared in this manner are mixed with Lipofectamine MessengerMAX at a ratio of 1:1 to form transfection complexes per the manufacturer's instructions. The mRNA transfection complexes are then added to the wells containing “aged” and “control” fibroblasts at doses of 5000 ng RNA per well, and transfection is allowed to proceed for 6 hours. Wells receiving reprogramming factor mRNA transfection complexes without mRNA encoding B18R serve as a control. After transfection is complete, the transfection medium is discarded and fresh medium is applied to the wells. mRNA is transfected every day, every other day, every three days, every four days, or every five days.

At 3, 4, 5, 6, and/or 7 days, cell viability and/or proliferation is evaluated using cell proliferation assays (WST-8 or MTT) assay per the manufacturer's instructions (Sigma Aldrich).

At 3, 4, 5, 6, and/or 7 days, cells are stained (immunofluorescence) to evaluate the expression of collagen IV, fibronectin and laminin as rejuvenation markers; vimentin as an aging marker, IFIT1, IFIT2, IFIT3, IL6, INFB, OAS1, PKR and TLR3 as cellular immune response markers; and LDH assay and AK assay to assess toxicity.

At 3, 4, 5, 6, and/or 7 days, cells are lysed and total RNA is collected and reverse-transcribed to cDNA. Real-time PCR is used to evaluate expression of collagen IV, fibronectin and laminin as rejuvenation markers; vimentin as an aging marker, IFIT1, IFIT2, IFIT3, IL6, INFB, OAS1, PKR and TLR3 as cellular immune response markers; and LDH assay and AK assay to assess toxicity.

Addition of mRNA encoding B18R results in higher translation efficiency and lower toxicity due to reduced type I interferon response as well as fewer transfections required and lower mRNA doses required. Higher translation efficiency, lower toxicity, and use of fewer transfections and lower mRNA dose provides lower toxicity and as a result, higher reprogramming efficacy and stronger cellular rejuvenation effects. Thus, addition of mRNA encoding B18R elicits higher cell viability and proliferation than without B18R mRNA. Addition of mRNA encoding B18R, provides up-regulation of cell rejuvenation markers and up-regulation of cell immune response than transfection without B18R, while toxicity, and aging markers are down-regulated with addition of B18R compared to the treatment without mRNA encoding B18R.

### Example 6

### Vectors with On-Off Switch for Expression of Reprogramming Factors

Monocistronic self-amplifying RNA molecules encoding the reprogramming factors OCT4 (O), SOX2 (S), KLF4 (K), c-MYC (M), LIN28 (L), NANOG (N), and GLIS1 (G) (each molecule encoding a single factor) are synthesized via in vitro transcription from plasmid DNA and purified. Each monocistronic mRNA molecule contains a 5′ cap, a 5′-UTR containing L7Ae regulatory sequence, a coding sequence for a single reprogramming factor, a 3′ UTR, and a polyA tail. In other conditions, polycistronic RNA molecules that each encode more than one factor are used. Human fibroblasts are obtained from Lonza and cultured in FGM-2 medium. An aging model is induced in the fibroblasts through treatment with TGF-beta at a concentration of 0.1-20 ng/ml for 3 days (“aged”), with untreated fibroblasts used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×106 cells/well and allowed to grow to 70% confluency in FGM-2 medium. Self-amplifying RNA molecules are prepared as naked RNA in nuclease-free H2O and then mixed together to provide reprogramming factor cocktails as follows: OSKMLN, OSKM, OSK, OSKG or other combinations of the reprogramming factors. or cocktails with other combinations of reprogramming factors (see abbreviations above). The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). To provide an on-off switch, L7Ae-containing mRNA is used. RNA cocktails prepared in this manner are mixed with Lipofectamine MessengerMAX at a ratio of 1:1 to form transfection complexes per the manufacturer's instructions. The RNA transfection complexes are then added to the wells containing “aged” and “control” fibroblasts at doses of 0.1-20 ng/ml ng RNA per well, and transfection is allowed to proceed for 6 hours. Wells receiving Lipofectamine alone serve as a control. After transfection is complete, the transfection medium is discarded and fresh medium is applied to the wells. Self-amplifying RNA is transfected once on Day 1, at the beginning of the experiment. L7Ae-containing mRNA is transfected to stop expression of self-amplifying RNA at 3, 4, 5, 6, or 7 days.

At 6, 7, 8, 9, 10, 11, 12, 13, or 14 days, cells are stained and analyzed by immunofluorescence to evaluate the expression of the reprograming factors; CD44, CD73 and CD105 as stemness markers; collagen 1A2, HSP47, Fibroblast-specific protein 1 (FSP1), a-Smooth muscle actin (α-SMA), Serpin Family H Member 1 (SERPINH1), CD44, prolyl 4-hydroxylase (P4HB), S100 calcium binding protein A4 (S100A4), Thy-1 Cell Surface Antigen (THY1) as lineage-specific markers; collagen IV, fibronectin and laminin as rejuvenation markers; and vimentin as an aging marker.

At 6, 7, 8, 9, 10, 11, 12, 13, or 14 days, cells are lysed and total RNA is collected and reverse-transcribed to cDNA. Real-time PCR is used to evaluate the expression of the reprograming factors; CD44, CD73 and CD105 as stemness markers; collagen 1A2, HSP47, FSP1, a-SMA, SERPINH1, CD44, P4HB, S100A4, THY1 as lineage-specific markers; collagen IV, fibronectin and laminin as rejuvenation markers; and vimentin as an aging marker.

L7Ae on-off switch mechanisms shut off expression of the reprograming factors at desired time points, reflected as decreased or undetectable expression of the reprogramming factors, whereas the reprogramming factor expression will continue in cells treated with self-replicating RNA without an on-off switch. All tested conditions will show rejuvenation and de-aging. However, continued expression of reprogramming factors by self-replicating RNA leads to increased stemness and a loss of cell identity and cell lineage. In contrast, using the on-off switch to shut off expression of the reprogramming factors after rejuvenation and de-aging through epigenetic reprogramming occur, but before loss of cell identity and cell lineage occur. Accordingly, stemness markers are not up-regulated and cell identity and cell lineage markers will not be down-regulated in on-off switch scenario.

### Example 7

### Polycistronic RNA for Expression of Reprogramming Factors

Polycistronic RNA molecules encoding the reprogramming factors OCT4 (O), SOX2 (S), KLF4 (K), c-MYC (M), LIN28 (L), NANOG (N), and GLIS1 (G) (each molecule encoding two, three, four, five, or six factors, for example LMK and OSK) are synthesized via in vitro transcription from plasmid DNA and purified. Each mRNA molecule contains a 5′cap, 5′-UTR, coding sequences for two, three, four, five, or six factors, an IRES element or 2A element before each coding sequence such that each gene has its own IRES or 2A element, a 3′ UTR, and a polyA tail. Human fibroblasts are obtained from Lonza and cultured in FGM-2 medium. An aging model is induced in the fibroblasts through treatment with TGF-beta at a concentration of 0.1-20 ng/ml for 3 days (“aged”), with untreated fibroblasts used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×106 cells/well and allowed to grow to 70% confluency in FGM-2 medium. Polycistronic RNA molecules are prepared as naked RNA in nuclease-free H2O and then mixed together to provide the full set of reprogramming factor combinations OSKMLN, OSKM, OSK, OSKG; or other combinations, for example, a polycistronic RNA encoding LMK could be mixed with polycistronic RNA encoding OSK. As a control, monocistronic mRNA, each encoding a single reprogramming factor, is used and mixed to provide OSKMLN, OSKM, OSK, OSKG, or other combinations. RNA cocktails prepared in this manner are mixed with Lipofectamine MessengerMAX at a ratio of 1:1 to form transfection complexes per the manufacturer's instructions. The RNA transfection complexes are then added to the wells containing “aged” and “control” fibroblasts at doses of 5000 ng RNA per well, and transfection is allowed to proceed for 6 hours. Wells receiving only vehicle (Lipofectamine messengerMAX) serve as a control. After transfection is complete, the transfection medium is discarded and fresh medium is applied to the wells. The polycistronic RNA is transfected every day, every other day, every three days, every four days, or every five days, as is the monocistronic mRNA.

At 6, 7, 8, 9, 10, 11, 12, 13, or 14 days, cells are collected and immunofluorescence is used to evaluate the expression of the reprogramming factors OCT4, SOX2, KLF4, c-MYC/GLIS1, LIN28, NANOG; CD44, CD73 and CD105 as stemness markers; collagen 1A2, HSP47, FSP1, a-SMA, SERPINH1, CD44, P4HB, S100A4, THY1 as lineage-specific markers; increased expression of collagen IV, fibronectin and laminin as rejuvenation markers; and vimentin an as aging marker.

At 6, 7, 8, 9, 10, 11, 12, 13, or 14 days, cells are lysed and total RNA is collected and reverse-transcribed to cDNA. Real-time PCR is used to evaluate the expression of the reprograming factors; CD44, CD73 and CD105 as stemness markers; collagen 1A2, HSP47, FSP1, a-SMA, SERPINH1, CD44, P4HB, S100A4, THY1 as lineage-specific markers; increased expression of collagen IV, fibronectin and laminin as rejuvenation markers; and vimentin as an aging marker.

Polycistronic RNA expression increases the likelihood of all reprogramming factors, or the minimum amount of factors required for effective epigenetic reprogramming, to be present in the same cell, thus leading to higher reprogramming efficiency as determined by higher numbers of cells showing rejuvenation or de-aging, or higher expression of rejuvenation markers and lower expression of aging markers, compared to monocistronic RNA expression. Also, use of independent IRES elements for each reprogramming factor allows the relative expression ratio of the factors to be equal (e.g., 1:1:1:1:1:1 for O:S:K:M:L:N) or tuned/adjusted (e.g., 2:1:1:1:1:1 for O:S:K:M:L:N), thus increasing the reprogramming efficiency. Additionally, expression of the reprogramming factors from polycistronic RNA does not result in increased stemness or loss of cell identity or lineage.

### Example 8

### Circular RNA for Expression of Reprogramming Factors

Circular RNA molecules encoding the reprogramming factors OCT4 (O), SOX2 (S), KLF4 (K), c-MYC (M), LIN28 (L), NANOG (N), and GLIS1 (G) (each molecule encoding a single reprogramming factor) are synthesized via in vitro transcription from plasmid DNA, circularized, and purified. Circular RNA molecules are produced using the Anabena intron-exon splicing strategy which consists of a fused partial intron at one end of the RNA and a partial exon at the other end RNA. Circularization by trans-esterification is carried out in presence of GTP and Mg2+ to produce circular RNA. Each mRNA molecule contains a IRES element, the coding sequence for a single reprogramming factor, and a 3′ UTR. Human fibroblasts are obtained from Lonza and cultured in FGM-2 medium. An aging model is induced in the fibroblasts through treatment with TGF-beta at a concentration of 0.1-20 ng/ml for 3 days (“aged”), with untreated fibroblasts used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×106 cells/well and allowed to grow to 70% confluency in FGM-2 medium. Circular RNA molecules are prepared as naked RNA in nuclease-free H2O and then mixed together to provide the full set of reprogramming factor combinations OSKMLN, OSKM, OSK, OSKG, or other combinations. The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). As a control, linear mRNA molecules, each encoding a single reprogramming factor, are used and mixed to provide OSKMLN, OSKM, OSK, OSKG, or other combinations. RNA cocktails prepared in this manner are mixed with Lipofectamine MessengerMAX at a ratio of 1:1 to form transfection complexes per the manufacturer's instructions. The RNA transfection complexes are then added to the wells containing “aged” and “control” fibroblasts at doses of 5000 ng RNA per well, and transfection is allowed to proceed for 6 hours. Wells receiving Lipofectamine MessengerMaX serve as a control. After transfection is complete, the transfection medium is discarded and fresh medium is applied to the wells. The circular RNA is transfected every day, every other day, every three days, every four days, or every five days, as is the linear mRNA.

At 6, 7, 8, 9, 10, 11, 12, 13, or 14 days, cells are lysed and total RNA is collected and reverse-transcribed to cDNA. Real-time PCR is used to evaluate the expression of the reprograming factors; CD44, CD73 and CD105 as stemness markers; collagen 1A2, HSP47, vimentin, FSP1, a-SMA, SERPINH1, CD44, P4HB, S100A4, THY1 as lineage-specific markers; increased expression of collagen IV, fibronectin and laminin as rejuvenation markers; and vimentin as an aging marker.

At 6, 7, 8, 9, 10, 11, 12, 13, or 14 days, cells are lysed and total RNA is collected and reverse-transcribed to cDNA. Real-time PCR is used to evaluate the expression of the reprograming factors; CD44, CD73 and CD105 as stemness markers; collagen 1A2, HSP47, FSP1, a-SMA, SERPINH1, CD44, P4HB, S100A4, THY1 as lineage-specific markers; increased expression of collagen IV, fibronectin and laminin as rejuvenation markers; and vimentin as an aging marker.

Use of circular RNA allows fewer transfections to be applied and lower RNA doses to be used when compared to conventional mRNA because of the persistence and lower immunogenicity of the circular RNA. The need for fewer transfections and lower RNA dose provides lower toxicity and higher reprogramming efficacy, leading to stronger cellular rejuvenation effects. Circular RNA provides higher cell viability and proliferation than conventional linear mRNA. Accordingly, up-regulation of cell rejuvenation markers and cell immune response is achieved with circular vs. linear mRNA, while toxicity, and aging markers are down-regulated with circular mRNA compared to treatment with linear mRNA.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A reprogramming factor encoding polynucleotide having at least 95% sequence identity to any one of SEQ ID NOs: 1-6 or 10.

2. A reprogramming factor protein or polypeptide encoded by a polynucleotide having at least 95% sequence identity to any one of SEQ ID NOs: 1-6 or 10.

3. An RNA vector, comprising:
one or more reprogramming factor polynucleotide sequences, wherein the one or more polynucleotide sequences consists of a polynucleotide having at least 95% sequence identity to any one of SEQ ID NOs: 1-6 or 10.

4. The RNA vector of claim 3, wherein the one or more reprogramming polynucleotide sequences encode a reprogramming protein or polypeptide with altered secondary or tertiary structure compared to wild-type reprogramming factor proteins or polypeptides.

5. The RNA vector of claim 4, wherein the reprogramming protein or polypeptide with altered structure triggers a reduced immune response, or exhibits altered activity or stability, compared to wild-type reprogramming factor proteins or polypeptides.

6. The RNA vector of claim 3, comprising from 5′ to 3′: (a promoter)-(a first polynucleotide sequence for a first reprogramming factor)-(a first reprogramming factor separating region)-(a second polynucleotide sequence for a second reprogramming factor)-(a second reprogramming factor separating region)-(a third polynucleotide sequence for a third reprogramming factor)-(optional additional polynucleotide sequences for optional additional reprogramming factors)-(optional additional separating regions)-(optional selectable marker)-(virus 3′UTR or a tail)-(optional selectable marker)-(optional promoter).

7. The RNA vector of claim 6, wherein the reprogramming factor separating regions each consist of one or more of the following: an IRES, a promoter and a self-cleaving peptide.

8. The RNA vector of claim 3, comprising a reprogramming factor selected from selected from the group consisting of Oct, Sox, Klf, Lin, Nanog, Myc and Glis.

9. The RNA vector of claim 3, comprising a reprogramming factor selected from the group consisting of OCT4, SOX2, KLF4, LIN28, NANOG, c-Myc, and Glis1.

10. The RNA vector of claim 9, wherein the reprogramming factor comprises OCT4.

11. The RNA vector of claim 10, wherein the OCT4 consists of a nucleotide sequence having at least 95% sequence identity to SEQ ID NO: 1.

12. The RNA vector of claim 9, wherein the reprogramming factor comprises SOX2.

13. The RNA vector of claim 12, wherein the SOX2 consists of a nucleotide sequence having at least 95% sequence identity to SEQ ID NO: 2.

14. The RNA vector of claim 9, wherein the reprogramming factor comprises C-Myc.

15. The RNA vector of claim 14, wherein the C-Myc consists of a nucleotide sequence having at least 95% sequence identity to SEQ ID NO: 3.

16. The RNA vector of claim 9, wherein the reprogramming factor comprises KLF4.

17. The RNA vector of claim 16, wherein the KLF4 consists of a nucleotide sequence having at least 95% sequence identity to SEQ ID NO: 4.

18. The RNA vector of claim 9, wherein the reprogramming factor comprises LIN28.

19. The RNA vector of claim 18, wherein the LIN28 consists of a nucleotide sequence having at least 95% sequence identity to SEQ ID NO: 5.

20. The RNA vector of claim 9, wherein the reprogramming factor comprises NANOG.

21. The RNA vector of claim 20, wherein the NANOG consists of a nucleotide sequence having at least 95% sequence identity to SEQ ID NO: 6.

22. The RNA vector of claim 6, wherein the first polynucleotide sequence comprises an OCT4 nucleotide sequence having at least 95% sequence identity to SEQ ID NO: 1, the second polynucleotide sequence comprises a SOX2 nucleotide sequence having at least 95% sequence identity to SEQ ID NO: 2, and the third polynucleotide sequence comprises a KLF4 nucleotide sequence having at least 95% sequence identity to SEQ ID NO: 4.

23. The RNA vector of claim 6, wherein the first polynucleotide sequence comprises a LIN28 sequence having at least 95% sequence identity to SEQ ID NO: 5, the second polynucleotide sequence comprises a NANOG sequence having at least 95% sequence identity to SEQ ID NO: 6, and the third polynucleotide sequence comprises a c-Myc sequence having at least 95% sequence identity to SEQ ID NO: 3.

24. The RNA vector of claim 6, wherein the first polynucleotide sequence, second polynucleotide sequence and third polynucleotide sequence are independently selected from the group consisting of nucleotides comprising at least 95% sequence identity to SEQ ID NOs: 1-6 and 10.

25. A method of treating a cell, tissue or organ in a subject in need thereof, comprising:
contacting the cell, tissue or organ with an RNA vector according claim 3, whereby said contacting achieves expression of the one or more reprogramming factors in the cell, tissue or organ to obtain a rejuvenated cell tissue or organ with retention of cellular identity.
